(19) United States
(12) Patent Application Publication
(10) Pub. No.: US 2022/0192733 A1
(43) Pub. Date: Jun. 23, 2022

(54) MINIMALLY INVASIVE SURGERY ABLATION CLAMP WITH CAM MECHANISM

(71) Applicant: AtriCure, Inc., Mason, OH (US)

(72) Inventors: Heidi KOETT, Loveland, OH (US); Salvatore PRIVITERA, Mason, OH (US); Matthew MONTI, Cincinnati, OH (US); Melissa HARGIS, Mason, OH (US); Kurtis KLINGENBERG, Mason, OH (US); Frank M. Fago, Mason, OH (US); Ruixin FAN, Mason, OH (US); Christoph GILLUM, Mason, OH (US)

(73) Assignee: AtriCure, Inc., Mason, OH (US)

(21) Appl. No.: 17/478,784

(22) Filed: Sep. 17, 2021

Related U.S. Application Data
(60) Provisional application No. 63/123,810, filed on Dec. 10, 2020.

(51) Int. Cl. A61B 18/14

(52) U.S. Cl. CPC A61B 18/1445; A61B 2018/00577

(57) ABSTRACT

Apparatus and systems include an electrosurgical instrument with a unique jaw configuration and gripping surfaces that allow tangential gripping and ablation of tissue in a minimally invasive procedure. The electrosurgical instrument can include a shaft and hinge coupling the shaft to first and second jaws. The first jaw can be an anchor for the second jaw. The jaws can have respective first portions and second portions offset at an angle from the first portions. Each jaw can also include electrodes on their inner surfaces facing the other jaw. Each jaw can also include textured gripping surfaces. The gripping surfaces can be included on at least a portion of the base of each jaw, as well as on at least a portion of the inner surfaces of the jaws.

### CROSS-REFERENCE TO RELATED APPLICATIONS

This application claims the benefit of priority to U.S. Provisional Application 63/123,810 filed Dec. 10, 2020 which is incorporated herein by reference in its entirety.

### TECHNICAL FIELD

This application relates to devices and methods for ablating tissue of a patient. More particularly, the application relates to devices and methods for ablating tissue that is difficult to grasp, such as for treating heart arrhythmias.

### INTRODUCTION

Radiofrequency (RF) clamps, such as bipolar clamps, are used in surgery for performing ablation of tissue. During some procedures, the surgeon creates encircling lesions around the superior vena cava (SVC) and inferior vena cava (IVC) as well as a connecting lesion along the crista terminalis (CT). For the SVC and IVC lesions, the vessel is encircled with the clamp by placing the distal jaw medially/posteriorly while the proximal jaw remains lateral/anterior. When the jaws are closed to create the ablation, a full thickness lesion is created circumferentially around the vessel. This is a typical approach applied during arrhythmia ablation procedures, such as pulmonary vein isolation during AF ablation. Current methods use RF clamps to create these types of lesions.

RF clamps can be used to ablate cardiac tissue by clamping the tissue between electrodes. Existing RF clamps can be used to encircle tissue and perform ablation. However, RF clamps typically have smooth, rounded surfaces that can be useful to prevent undesired snagging during a surgical procedure. While existing RF clamps with such surfaces are useful in encircling tissue, such as arteries and the like, problems arise when attempting to use existing RF clamps to ablate through folded sections of intact sections of tissue, such as on the atrium of a heart (e.g., the crista terminalis), due to the geometries of the RF clamp relative to the layout of the target tissue, such as a line on the crista terminalis.

Work-arounds to address these issues leave opportunity for improvement. For example, using endoscopic surgical graspers to grip the tissue and pull it into the jaws of the RF clamp, or using an endoscopic kittner to invaginate the tissue into the jaws of the RF clamp, still subject the operator/surgeon to risks of slipping and otherwise sub-optimal gripping. Moreover, it introduces additional devices into a small, limited surgical space and/or requires the introduction of multiple different devices at different times into the surgical space. Another approach relies upon performing dissection and isolation of the right pulmonary veins, which is a challenging technique even for skilled surgeons.

Therefore, there exists a need to develop devices and methods that can create a tangential lesion along intact cardiac atrial tissue during a closed chest, beating heart procedure without the use of an accessory such as an endoscopic kittner or grasper. There also exists a need for such a procedure without dissection of the pulmonary veins.

### BRIEF SUMMARY OF THE INVENTION

The following summarizes some aspects of the present disclosure to provide a basic understanding of the discussed technology. This summary is not an extensive overview of all contemplated features of the disclosure and is intended neither to identify key or critical elements of all aspects of the disclosure nor to delineate the scope of any or all aspects of the disclosure. Its sole purpose is to present some concepts of one or more aspects of the disclosure in summary form as a prelude to the more detailed description that is presented later.

For example, in a variation of the disclosure, a tissue grabbing apparatus can comprise a first jaw comprising a first jaw member approximately parallel to a shaft of the tissue grabbing apparatus and a second jaw member offset from approximately parallel to the shaft, the first jaw can further comprise a first surface having a base of the first jaw member and a base of the second jaw member, and a second surface can further comprise an inner surface of the first jaw member and the second jaw member, wherein the first jaw can further comprise a first pair of electrodes extending along the second surface including at least part of the first jaw member and the second jaw member, and a first grasping surface can further comprise a first grasping pattern extending along at least part of the second surface and along at least part of the first surface. The tissue grabbing apparatus can further comprise a second jaw comprising a third jaw member at an angle to the shaft, and a fourth jaw member offset from parallel to the third jaw member while the tissue grabbing apparatus is in an open position, the second jaw can further comprise a third surface having a base of the third jaw member and a base of the fourth jaw member, and a fourth surface can face the second surface and can further comprise an inner surface of the third jaw member and the fourth jaw member, wherein the second jaw further can further comprise a second pair of electrodes extending along the fourth surface including at least part of the third jaw member and the fourth jaw member, and a second grasping surface can further comprise a second grasping pattern extending along at least part of the fourth surface and along at least part of the third surface. The first grasping pattern and the second grasping pattern can be the same pattern, different patterns, or can have mirrored patterns. The tissue grabbing apparatus can further comprise a hinge assembly connecting the first and second jaws and can be configured to move the second jaw relative to the first jaw in a direction transverse to an axis parallel to the shaft between the open position and a closed position where the second jaw is approximately parallel to the first jaw and the shaft.

The first jaw can further comprise a fifth surface interfacing between the first surface and the second surface. The fifth surface can comprise the first grasping pattern. The second jaw can further comprise a sixth surface interfacing between the third surface and the fourth surface. The sixth surface can comprise the second grasping pattern. The first jaw member and second jaw member can meet at a first apex comprising an angle. The third jaw member and the fourth jaw member can meet at a second apex comprising an angle. The hinge assembly can comprise at least one of a bell crank hinge mechanism, a rack-and-pinion hinge mechanism, or a cam hinge mechanism. The first jaw and the second jaw can be removeable and replaceable from respective distal ends of the hinge assembly. The tissue grabbing apparatus can comprise an atraumatic tip coupled to a distal end of the first jaw. The atraumatic tip can comprise a blue color, red color, or white color for increasing visibility of the tip in contrast with surrounding tissues.

The tissue grabbing apparatus can further comprise an angle of about 60 degrees between an outer surface of the second jaw member and the shaft. The tissue grabbing apparatus can further comprise a gripping feature on an outer surface of the first jaw and an outer surface of the second jaw. The gripping feature can comprise an abrasive coating. The gripping feature can comprise a pattern, the pattern can be machined, etched, coined, knurled, or stamped into the first jaw and the second jaw. The tissue grabbing apparatus can further comprise an insulator component. The insulator component can either be removably detached from the first jaw or permanently attached to the first jaw by mechanical means, such as snap fits. The jaw assembly can comprise a cam assembly. The cam assembly can be configured to slide axially within the jaw assembly to move the second jaw relative to the first jaw. The second jaw can comprise at least one bearing. The at least one bearing can be configured to slide within a track of the cam assembly. The hinge assembly can have a fixed angle. The fixed angle can be about 30 degrees.

In another variation of the disclosure, a surgical clamp system for grabbing and ablating tissue can comprise a handle. The surgical clamp system can further comprise an elongated shaft comprising a proximal end coupled to the handle and a distal end. The surgical clamp system can further comprise a jaw assembly comprising a first jaw comprising a first surface having a base of the first jaw, a second surface can comprise an inner surface of the first jaw, a first electrode extending along the second surface, and a first grasping surface can comprise a first grasping pattern extending along at least part of the first surface and at least part of the second surface, and a second jaw comprising a third surface having a base of the second jaw, a fourth surface comprising an inner surface of the second jaw, a second electrode extending along the fourth surface, and a second grasping surface comprising a second grasping pattern extending along at least part of the fourth surface and at least part of the third surface. The surgical clamp system can further comprise a hinge assembly coupled to the distal end of the shaft, connecting the first and second jaws and configured to move the second jaw relative to the first jaw in a direction transverse to an axis parallel to the shaft.

The surgical clamp system can further comprise a plunger coupled via the handle and shaft to the hinge assembly. Actuation of the plunger can cause the second jaw to move in the direction. The surgical clamp system can further comprise a trocar slidably coupled around the shaft and configured to provide an insufflation seal to the surgical clamp system after insertion of the jaw assembly into a patient. The surgical clamp system can further comprise a seal within the shaft configured to provide an internal insufflation seal to the surgical clamp system in use. The seal can comprise a plug component. The plug component can reduce a cross-sectional area inside the shaft. The plug and mating components may be configured such that a seal is created when the clamp is in completely open or closed positions, but a gas flow path is present when the clamp is partially open for purposes of sterilization with EtO gas. The surgical clamp system can further comprise an atraumatic tip coupled to a distal end of the first jaw. The hinge assembly can comprise at least one of a bell crank hinge mechanism, a rack-and-pinion hinge mechanism, or a cam hinge mechanism. The first grasping surface and the second grasping surface can each comprise a plurality of pyramid-shaped teeth each culminating in a flat surface before reaching a pyramid tip. Each pyramid-shaped tooth of the first and second grasping surface can be biased in a direction based on a force profile of the surgical clamp system.

In another aspect of the disclosure, a tissue grabbing apparatus can comprise a first jaw comprising a first surface having a base of the first jaw, a second surface comprising an inner surface of the first jaw, a first electrode extending along the second surface, and a first grasping surface comprising a first grasping pattern extending along at least part of the first surface and at least part of the second surface. The tissue grabbing apparatus can further comprise a second jaw coupled to the first jaw via a hinge assembly, the second jaw comprising a third surface having a base of the second jaw, a fourth surface comprising an inner surface of the second jaw, a second electrode extending along the fourth surface, and a second grasping surface comprising a second grasping pattern extending along at least part of the fourth surface and at least part of the third surface, wherein the first grasping surface and the second grasping surface each comprises a plurality of pyramid-shaped teeth each culminating in a flat surface before reaching a pyramid tip and comprising a pitch between centerpoints of each pyramid-shaped tooth of between one tenth of an inch and five tenths of an inch and a height of each pyramid-shaped tooth of between one tenth of an inch and two tenths of an inch. The teeth can be cuboidially-shaped. Each of the plurality of cuboidially-shaped teeth can culminate in a pointed, flat, chamfered or filleted surface before reaching a pyramid tip.

Each pyramid-shaped tooth of the first and second grasping surfaces can be biased in a direction based on a force profile of the tissue grabbing apparatus. The plurality of pyramid-shaped teeth can be in a square pyramid configuration. The first grasping surface can terminate with a first point at an edge of the first grasping surface adjacent to an edge of the second grasping surface. The second grasping surface terminates with a second point at the edge of the second grasping surface adjacent the edge of the first grasping surface. The hinge assembly can be configured to move the second jaw relative to the first jaw in a direction transverse to an axis parallel to a shaft of the tissue grabbing apparatus between an open position and a closed position where the second jaw is approximately parallel to the first jaw and the shaft. The first and second grasping surfaces can be configured to grab tissue as the second jaw moves to the closed position.

In another aspect of the disclosure, a method of operating an electrosurgical instrument can include inserting first and second jaws of the electrosurgical instrument into a body cavity of a patient, the first and second jaws including respective inner surfaces with respective electrodes and respective grasping surfaces on at least portions of the respective inner surfaces and bases of the first and second jaws. The method can further comprise grasping tissue with at least portions of the respective grasping surfaces along a crista terminalis (CT) line. The method can further comprise ablating the grasped tissue along the CT line. The method can further comprise manipulating and encircling a superior vena cava (SVC) with the first and second jaws, and ablating tissue of the SVC at a first end of the CT line. The method can further comprise manipulating and encircling an inferior vena cava (IVC) with the first and second jaws, and ablating tissue of the IVC at a second end of the CT line.

Other aspects, features, and embodiments of the present disclosure will become apparent to those of ordinary skill in the art, upon reviewing the following description of specific, example embodiments of the present disclosure in conjunction with the accompanying figures. While features of the present disclosure can be discussed relative to certain embodiments and figures below, all embodiments of the present disclosure can include one or more of the advantageous features discussed herein. In other words, while one or more embodiments can be discussed as having certain advantageous features, one or more of such features can also be used in accordance with the various embodiments of the disclosure discussed herein. In similar fashion, while example embodiments can be discussed below as device, system, or method embodiments it should be understood that such example embodiments can be implemented in various devices, systems, and methods.

### BRIEF DESCRIPTION OF THE DRAWINGS

The present disclosure is best understood from the following detailed description when read with the accompanying figures.

FIG. 1 illustrates an exemplary radiofrequency (RF) tissue ablation system according to one or more aspects of the present disclosure.

FIG. 2A illustrates an exemplary perspective view of an RF tissue ablation clamp according to one or more aspects of the present disclosure.

FIG. 2B illustrates an exemplary exploded view of an RF tissue ablation clamp according to one or more aspects of the present disclosure.

FIG. 2C illustrates an exemplary perspective view of an insufflation seal according to one or more aspects of the present disclosure.

FIG. 3A illustrates an exemplary perspective view of RF tissue ablation jaws of an RF tissue ablation clamp according to one or more aspects of the present disclosure.

FIG. 3B illustrates an exemplary, cross-sectional view of RF tissue ablation jaws of an RF tissue ablation clamp according to one or more aspects of the present disclosure.

FIG. 3C illustrates an exemplary view of insulator components coupled to the RF tissue ablation jaws according to one or more aspects of the present disclosure.

FIG. 4A illustrates an exemplary top plan view of an RF tissue ablation clamp according to one or more aspects of the present disclosure.

FIG. 4B illustrates an exemplary bottom plan view of an RF tissue ablation clamp according to one or more aspects of the present disclosure.

FIG. 4C illustrates an exemplary side view of an RF tissue ablation clamp according to one or more aspects of the present disclosure.

FIG. 4D illustrates an exemplary top view of a cross-section of the RF tissue ablation clamp of FIG. 4C according to one or more aspects of the present disclosure.

FIG. 4E illustrates an exemplary side view of an RF tissue ablation clamp according to one or more aspects of the present disclosure.

FIG. 5A illustrates an exemplary bottom perspective view of RF tissue ablation jaws of an RF tissue ablation clamp in a first position according to one or more aspects of the present disclosure.

FIG. 5B illustrates an exemplary bottom view of RF tissue ablation jaws of an RF tissue ablation clamp in a second position according to one or more aspects of the present disclosure.

FIG. 5C illustrates an exemplary cross-sectional view of a gripping surface of an RF tissue ablation jaw of an RF tissue ablation clamp according to one or more aspects of the present disclosure.

FIGS. 5D-5L illustrate teeth configuration alternatives of RF tissue ablation jaws of an RF tissue ablation clamp according to one or more aspects of the present disclosure.

FIG. 6A illustrates an exemplary front view of RF tissue ablation jaws with a hinge of an RF tissue ablation clamp according to one or more aspects of the present disclosure.

FIGS. 6B-6D illustrate exemplary cross-sectional views of RF tissue ablation jaws with hinge configuration alternatives of an RF tissue ablation clamp according to one or more aspects of the present disclosure.

FIGS. 6E-6H illustrate exemplary cross-sectional views of a sliding cam mechanism for movement of the RF tissue ablation jaws according to one or more aspects of the present disclosure.

FIG. 7A illustrates an exemplary top plan view of an RF tissue ablation clamp with interchangeable jaws according to one or more aspects of the present disclosure.

FIGS. 7B-7D illustrate exemplary views of interchangeable jaw configurations for an RF tissue ablation clamp according to one or more aspects of the present disclosure.

FIGS. 8A-8C illustrate exemplary side views of alternative atraumatic bumper tip configurations for an RF tissue ablation clamp according to one or more aspects of the present disclosure.

FIG. 9 illustrates an exemplary side view of an RF tissue ablation clamp with trocar according to one or more aspects of the present disclosure.

FIG. 10A illustrates an exemplary cross-sectional view of a shaft of an RF tissue ablation clamp according to one or more aspects of the present disclosure.

FIGS. 10B-10C illustrate exemplary alternatives for shaft seals for an RF tissue ablation clamp according to one or more aspects of the present disclosure.

FIG. 11 illustrates an exemplary side view of a heart showing candidate ablation locations with an exemplary RF tissue ablation clamp according to one or more aspects of the present disclosure.

FIG. 12 illustrates an exemplary view of RF tissue ablation jaws side-clamping tissue according to one or more aspects of the present disclosure.

FIG. 13 illustrates an exemplary top perspective view of a patient showing insertion of the RF tissue ablation clamp into the chest cavity according to one or more aspects of the present disclosure.

FIG. 14 illustrates an exemplary method of using an exemplary RF tissue ablation clamp in an ablation procedure according to one or more aspects of the present disclosure.

### DETAILED DESCRIPTION

The detailed description set forth below, in connection with the appended drawings, is intended as a description of various configurations and is not intended to represent the only configurations in which the concepts described herein can be practiced. The detailed description includes specific details for the purpose of providing a thorough understanding of the various concepts. However, it will be apparent to those skilled in the art that these concepts can be practiced without these specific details. In some instances, well-known structures and components are shown in block diagram form in order to avoid obscuring such concepts.

All examples and illustrative references are non-limiting and should not be used to limit the claims to specific implementations and embodiments described herein and their equivalents. For simplicity, reference numbers can be repeated between various examples. This repetition is for clarity only and does not dictate a relationship between the respective embodiments. Finally, in view of this disclosure, particular features described in relation to one aspect or embodiment can be applied to other disclosed aspects or embodiments of the disclosure, even though not specifically shown in the drawings or described in the text.

Various embodiments include an electrosurgical instrument with a unique jaw configuration, with corresponding unique gripping surfaces, that allows tangential gripping and ablation of tissue, such as epicardial tissue in a minimally invasive procedure. The electrosurgical instrument can include a handle, shaft extending from the handle, and hinge coupling the shaft to a pair of jaws. The jaws can include a first jaw generally in a same plane as a plane of the shaft, and a second jaw offset at an angle from the first jaw. The first jaw can stay in place and serve as an anchor for the second jaw. The jaws can both have a first portion and a second portion offset at an angle from the first portion. Each jaw can also include electrodes on their inner surfaces, that is on the surfaces that face the other jaws. This allows the jaws to clamp tissue between them and ablate the tissue thus clamped.

Each jaw can also include textured gripping surfaces. The gripping surfaces can, in some examples, be formed with a cross-serration pattern, such as square pyramidal shapes with a predetermined pitch between peaks (e.g., on the order of 26 hundredths of an inch), a predetermined height (e.g., on the order of 13 hundredths of an inch). In some examples, the tips of each pyramidal shape can be flat. Thereby, the gripping surfaces can be able to grip and retain tissue while still providing a better atraumatic result, while also increasing the surface area for gripping with the jaws. The gripping surfaces can be included on at least a portion of the base of each jaw, as well as on at least a portion of the inner surfaces of the jaws. With the unique gripping surfaces and jaw configuration, a lesion can be formed along the Crista Terminalis (CT). The same electrosurgical instrument can be used to form encircling ablations on the superior vena cava (SVC) and inferior vena cava (IVC).

Benefits include creating a lesion set with the electrosurgical instrument along intact cardiac atrial tissue during a closed chest, beating heart procedure without use of an accessory such as an endoscopic kittner or dissection of the pulmonary veins. This can be achieved in response to the specific jaw angle of the electrosurgical instrument, as well as in response to the textured gripping surfaces of the jaws which facilitate tissue capture and retention in a tangential orientation.

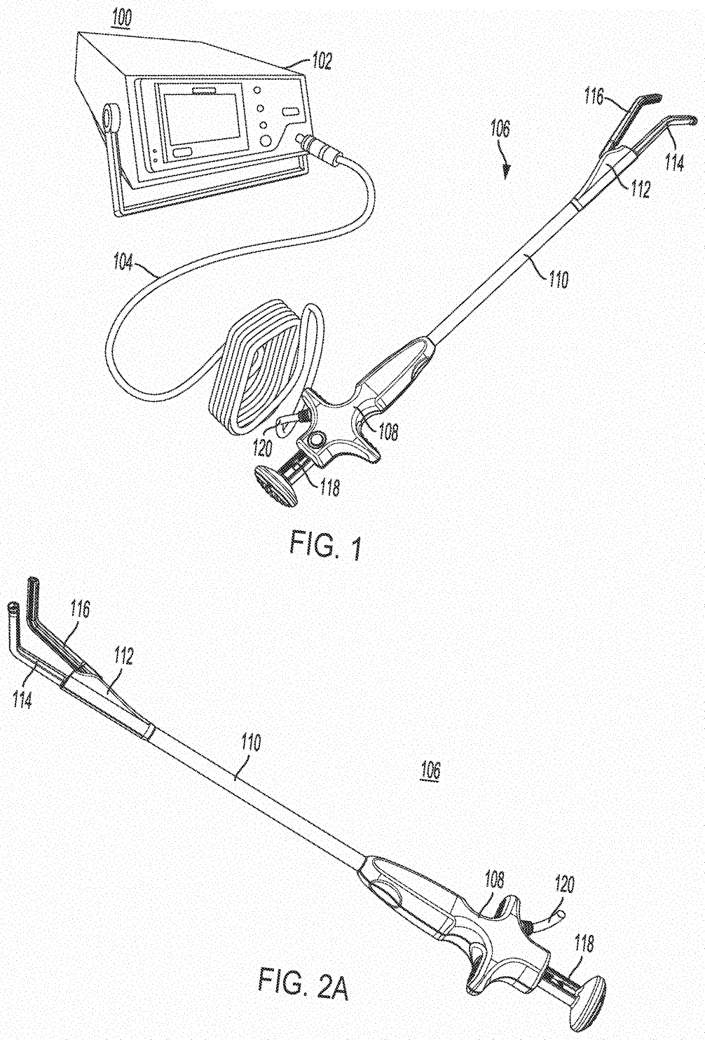

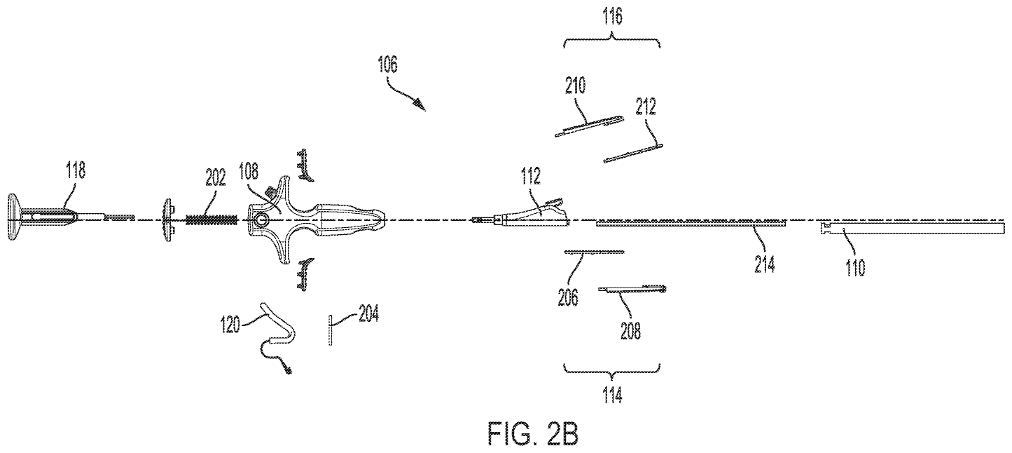

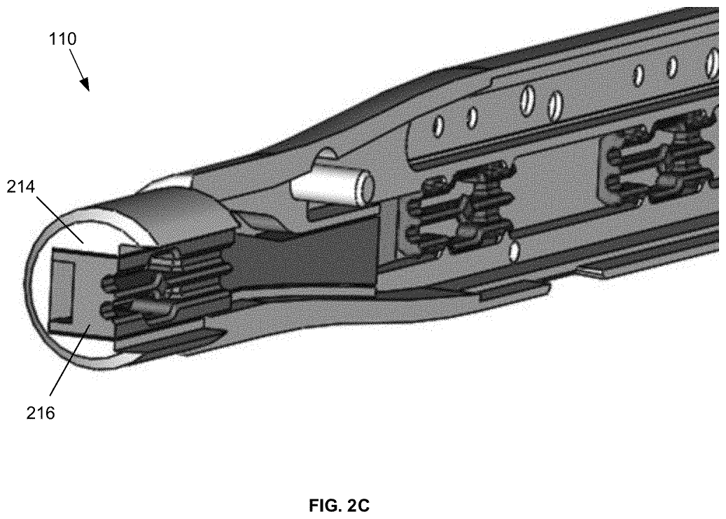

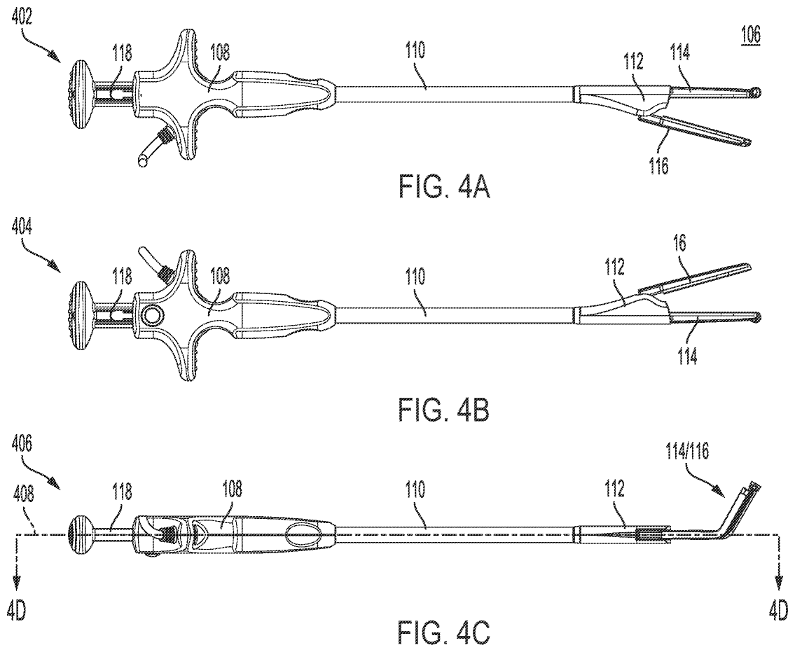

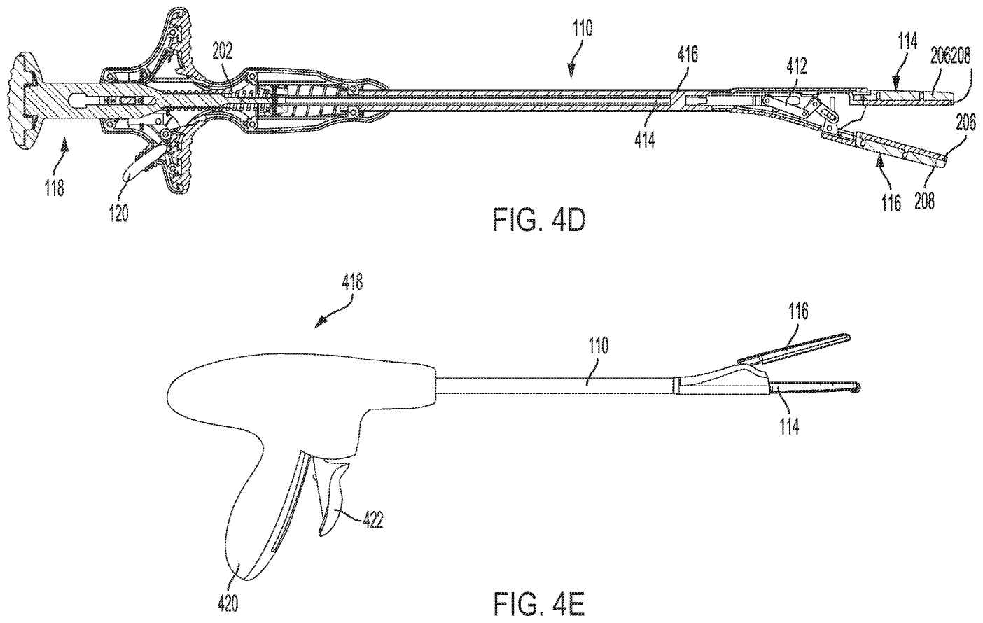

FIG. 1 illustrates an example of a tissue ablation system in the form of a bipolar electrosurgical system 100 having an electrosurgical instrument 106 coupled to an energy generator, e.g., an RF generator 102. Electrosurgical instrument 106, as illustrated in FIG. 1 and further in FIG. 2A and FIGS. 4A-4E, can include a handle 108, an elongated longitudinal shaft 110 extending therefrom, and an end effector for clamping and heating tissue therebetween. In some examples, the electrosurgical instrument 106 can be used in minimally invasive procedures, such as intercostal or subxiphoid approaches to cardiac tissue targeted for ablation. Alternatively, the electrosurgical instrument 106 can be used in open procedures where the ablation site is directly viewable by the surgeon.

The illustrated end effector has first jaw 114 and second jaw 116, in an opposed configuration for tangentially clamping tissue therebetween. The first and second jaws 114, 116 are shown spaced apart for the reception of tissue therebetween, with at least one of the first and distal jaws 114, 116 movable to clamp tissue therebetween. For example, the first jaw 114 can be in a fixed position relative to the elongated longitudinal shaft 110 and the second jaw 116. In particular, at least a proximal portion of the first jaw 114 that is attached to the elongated longitudinal shaft 110 via the hinge mechanism 112 can have an axis generally in alignment with the long axis of the elongated longitudinal shaft 110 (e.g., a first jaw member in some examples). As illustrated, the first jaw 114 can further include a distal portion at an angle out of alignment from the long axis of the elongated longitudinal shaft 110 (e.g., a second jaw member in some examples). In some examples, the angle can be in the range of 40-75 degrees, for example 60 degrees. Accordingly, a complementary angle between the long axis of the elongated longitudinal shaft 110 and an inner surface of the first and second jaws 114, 116 can be in the range of 105-140 degrees, for example 120 degrees.

Further, the second jaw 116 can be spaced apart from the first jaw 114. In some embodiments, the second jaw 116 can be attached to the elongated longitudinal shaft 110 via the hinge mechanism 112 at an axis offset from the long axis of the elongated longitudinal shaft 110. For example, at least a proximal portion of the second jaw 116 attached via the hinge mechanism 112 can be offset at an angle of between 10 and 45 degrees from the long axis of the elongated longitudinal shaft 110 (e.g., a third jaw member in some examples). Other angle offsets are possible as well while maintaining the general configuration as illustrated in FIG. 1 (or other figures illustrated and discussed herein). The second jaw 116 can likewise have a distal portion at an angle from the proximal portion of the second jaw 116 of equal magnitude to the angle of the offset of the distal portion of the first jaw 114 (e.g., a fourth jaw member in some examples).

To facilitate movement of the jaws 114, 116 to clamp tissue therebetween, first and second jaws 114, 116 can be operably coupled to a closure trigger. The closure trigger is illustrated in FIG. 1 as plunger 118 extending proximally from the handle 108 such that it is operable with one hand so that distal movement of plunger 118 towards the handle 108 can bring the first and second jaws 114, 116 together. The plunger 118 is illustrated via multiple perspectives in FIGS. 1-4D. In some other examples, the closure trigger can alternatively be configured according to a pistol/trigger style model. An example of this is illustrated in FIG. 4E.

In FIG. 4E, an alternative handle 418 is illustrated that includes a grip 420 with trigger 422, which would replace the handle 120 and plunger 118 illustrated by example in FIGS. 1-4D. The grip 420 can be, for example, in the form of a pistol grip. The grip 420 can encapsulate actuation and force control mechanisms to control movement of the jaw 116 (and/or jaw 114), as well as providing for grounding of the shaft components by means of a conductive shaft pin. In order to move the drive jaws 114, 116 between open and closed positions, the grip 420 can include a lever comprising, or controlled by, the trigger 422. The trigger 422 can be pivotally mounted on a support member within the grip 420. The grip 420 can further include a lever spring biasing the trigger 420 to its open position. Further, in embodiments using an alternative handle 418, the electrosurgical instrument 106 can be configured with inline rotation to allow tissue to be approached with the jaws 114, 116 in different orientations.

For example, where the first jaw 114 is fixed relative to the second jaw 116, the movement of the plunger 118 towards the handle 108 can cause the second jaw 116 to move via the hinge mechanism 112 towards the first jaw 114 until the jaws 114, 116 are pressed together either completely or as much as desired to maintain pressure on tissue grasped therebetween. This movement can be facilitated by a connection through the lumen 414 and/or 416 (FIG. 4D) that couples the plunger 118 to at least the jaw 116 via the hinge mechanism 112, for example via linkage 412 (FIG. 4D) to the jaw 116.

For example, where the tissue is heart tissue, a surgeon can apply normal force to a region (such as Waterston's Groove), and press the plunger 118 which sweeps the second jaw 116 along the surface of the tissue (such as the surface of the right atrium) to create a tangential bite of tissue for ablation between the first and second jaws 114, 116. Likewise, proximal movement of plunger 118 away from the handle 108 can move the first and second jaws 114, 116 apart. The present invention is not limited to the particular mechanism for moving the jaw(s) and an example of such a mechanism can be found in U.S. Pat. Nos. 6,923,806 and 7,291,161, both of which are incorporated by reference herein.

According to embodiments of the present disclosure, electrodes such as bidirectional ablation electrodes can be placed in a spaced-part relationship on the inner sides of the respective jaws. These inner sides of the jaws 114, 116 are the sides of the respective jaws that face the other, such that the electrodes are in a spaced-apart relation in sufficient proximity to the tissue to be ablated so that, upon activation each energy source creates an energy field in the tissue to be ablated. The energy sources (e.g., electrodes) are spaced such that the energy fields created by at least one of the activated sources partially overlaps with the energy field created by one or more of the other energy sources. The energy sources can further be alternately activated and deactivated, so that a substantially constant energy field results where the energy fields created by at least two of the energy sources overlap. While the energy sources are preferably RF energy sources, other energy sources, such as microwave, can be used.

Specifically, two or more pairs of opposed electrodes can be located in first and second jaws 114, 116 of the instrument 106. The electrodes can be operably coupled to the RF generator 102 by a cable 104 through the port 120 on the handle 108. The generator 102 can be operator-actuated by a switch, such as a foot switch extending therefrom. A control system/RF generator for providing RF energy and for use in the system of the present invention is shown in the application U.S. Pat. No. 8,603,083, which is incorporated herein by reference. The RF energy monitored impedance delivered to the electrode pairs can be based, at least in part, on the monitored impedance of the tissue to be ablated as it is held between the jaws of the instrument. To this end, the controller can monitor or sense voltage and/or currents associated therewith, calculating or deriving the impedance of the tissue between the electrodes of at least one of the pairs of opposed electrodes and between at least two of the pairs of opposed electrodes. The ablation can continue until the calculated impedance indicates that the lesion or ablation line is transmural (or fully through the tissue thickness).

FIG. 2B illustrates an exemplary exploded view of a tissue ablation system according to one or more aspects of the present disclosure. Specifically, FIG. 2B illustrates an exploded view of the electrosurgical instrument 106 introduced in FIG. 1. As shown, the plunger 118 can be coupled to the handle 108 via a spring 202. While referred to as a spring 202, other mechanisms can be used instead to provide biasing force. The spring 202 can provide a biasing force in a proximal direction away from the handle 108. This can correspond to the jaws 114, 116 being biased in an open position, with the jaw 116 being at an angle relative to the long axis of the elongated longitudinal shaft 110 and the jaw 114 being generally in alignment with the long axis of the elongated longitudinal shaft 110. Grips 203 provide surfaces for a user to grasp while holding the handle 108. They can be formed of a variety of materials, such as silicone, other plastic, and/or rubber material to name a few examples. The grips 203 can be formed with patterns, ridges, grooves, etc. That provide a textured surface for ease of gripping by a user.

The elongated longitudinal shaft 110 can couple to the handle 108 by a pin 204. While illustrated as a single pin 204, other or additional mechanism(s) can be used to attach (either separably or permanently) the elongated longitudinal shaft 110 with the handle 108 for operation. The elongated longitudinal shaft 110 can be hollow, with a lumen inside. This can be further seen by reference to FIG. 4D, which illustrates an exemplary top view of a cross-section 408 (FIG. 4C) of the electrosurgical instrument 106 of FIGS. 1-4C according to one or more aspects of the present disclosure. As shown in FIG. 4D, the elongated longitudinal shaft 110 includes a lumen 416. Referencing FIG. 2B again, the elongated longitudinal shaft 110 can also include an inner shaft 214 extending within the lumen 416 (FIG. 4D) along the inner length of the elongated longitudinal shaft 110 from the distal end of the handle 108 to the proximal end of the hinge mechanism 112. The inner shaft 214 can likewise be hollow with a lumen 414 (FIG. 4D) with sufficient diameter to allow one or more electrical leads to connect the electrodes of the electrosurgical instrument 106 with the RF generator 102 (FIG. 1) via the port 120. Or, alternatively, the inner shaft 214 can be open-sided, such as in the shape of a tray with one or two sides absent (illustrated, for example, in FIG. 10A as shaft 1002).

Still referring to FIG. 2B, in some embodiments the inner shaft 214 can also accommodate an insufflation seal to prevent loss of air pressure (such as carbon dioxide) used for insufflation of a body cavity during an operation. The insufflation seal can, in some embodiments, be a form of plug at a point along the length of the inner shaft 214 (or multiple plugs at different points along the length of the inner shaft 214) that can reduce a cross-sectional area inside the shaft. In other embodiments, the insufflation seal can be an elongate member that extends along a length of the inner shaft 214, with multiple flexible members extending from a center of the elongated member that, together, impede or otherwise prevent loss of air pressure from the body cavity. The flexible members can be, for example, bristles or other elements that together impede or stop air flow from passing through them.

FIG. 2C illustrates a cross-sectional view of the insufflation seal 216. The insufflation seal 216 can minimize loss of CO2 pressure through the device when insufflation is employed by incorporating the insufflation seal 216 to reduce the cross-sectional area inside the shaft of the clamp. During closed-chest, minimally invasive procedures, insufflation can be used for space creation and visualization within the thoracic cavity, which can enable flow of CO2 through the device and subsequent loss of pressure in the cavity throughout the procedure.

As noted with respect to the figures above and further below, each of the jaws 114, 116 can include energy sources such as electrodes on their mutually facing surfaces. As shown in the exploded view of FIG. 2B, the first jaw 114 includes a support surface 206 which can constitute an outer surface of the jaw 114 that faces away from the inner surface of the jaw 116. The first jaw 114 can also include pad surface 208 which can constitute an inner surface of the jaw 114 that faces towards the inner surface of the jaw 116. In like manner, the second jaw 116 can include a support surface 210 that faces away from the inner surface of the first jaw 114, and a pad surface 212 that constitutes an inner surface of the jaw 116 that faces towards the inner surface of the jaw 114.

For example, the pad surfaces 208, 212 can be connected with the respective support surfaces 206, 210 via adhesive, mechanical linkage(s), and/or other mechanisms. The support surfaces 206, 210 can be formed out of a metal, such as stainless steel. Alternatively, the support surfaces 206, 210 can be formed via injection molding. In some examples, the pad surfaces 208, 212 can be permanently connected to the respective support surfaces 206, 210 (or, alternatively, the pad surface and the support surface can be formed as one piece). In other examples, the pad surfaces 208, 212 can be releasably connected to the support surfaces 206, 210 and can be removed and/or swapped out with another pad surface for use in the electrosurgical instrument 106. Electrodes on the jaw 114 can be integrated with the pad surface 208 or attached to the pad surface 208 (similarly with respect to the electrodes on the jaw 116 and the pad surface 212).

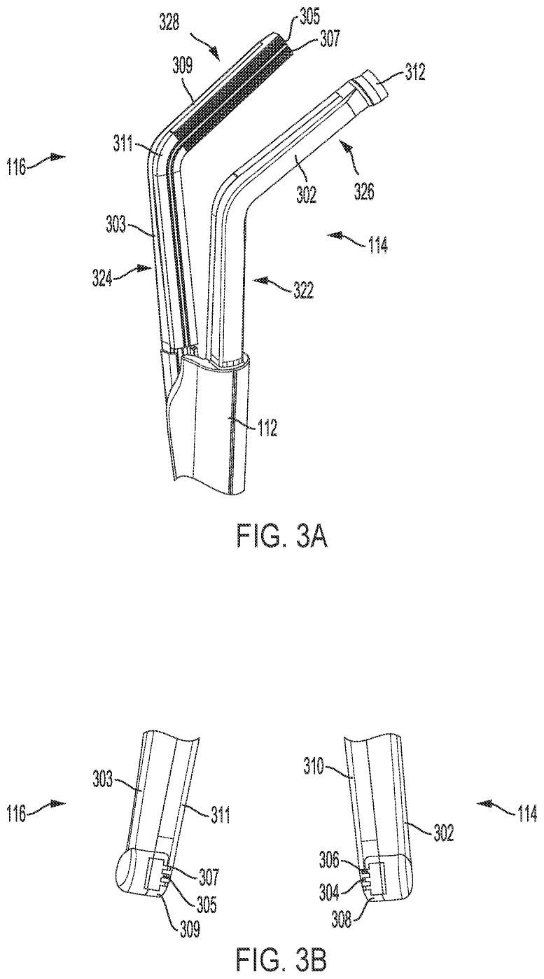

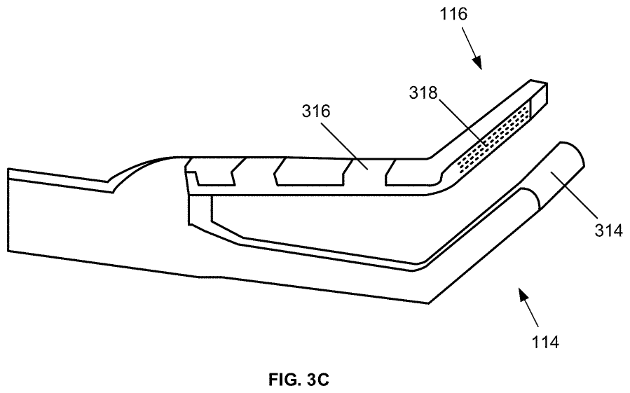

FIGS. 3A and 3B provide closer views of RF tissue ablation jaws, including of the electrodes on the jaws, of an RF tissue ablation clamp, such as the electrosurgical instrument 106 introduced in FIG. 1. As illustrated, the jaws 114, 116 include proximal portions that are generally parallel to a plane along which the elongated longitudinal shaft 110 extends. For example, jaw 114 can include proximal portion 322 and jaw 116 can include proximal portion 324. The jaws 114, 116 further have distal portions that are set at an angle to the plane in which the proximal portions are situated. For example, jaw 114 can include distal portion 326 and jaw 116 can include distal portion 328. This angle configuration can also be viewed with reference to FIG. 4C, which provides a side view 406 of the electrosurgical instrument 106. As an example, the distal portions of the jaws 114, 116 can be offset by a range of 110 degrees to 130 degrees from the plane in which the proximal portions are illustrated. As a specific example, the offset can be 120 degrees. This can provide an optimal alignment with the CT from a right inferior video-assisted thoracic surgery (VATS) port.

The proximal portions 322, 324 of jaws 114, 116 can have a length in the range of about 1.50-1.65in, for example 1.57 inches. The distal portions 326, 328 of jaws 114, 116 can have a length in the range of 0.7-0.9in, for example 0.79 inches.

As further shown in FIG. 3A, the jaw 116 can include electrodes 304 and 306. FIG. 3B (which provides a cross-sectional view of the distal ends of both jaws 114, 116 while in an open position) illustrates electrodes 304 and 306 on both jaws 114 and 116, meant to signify that both jaws have respective electrodes (not that they are the same electrodes or share the same control signaling as each other).

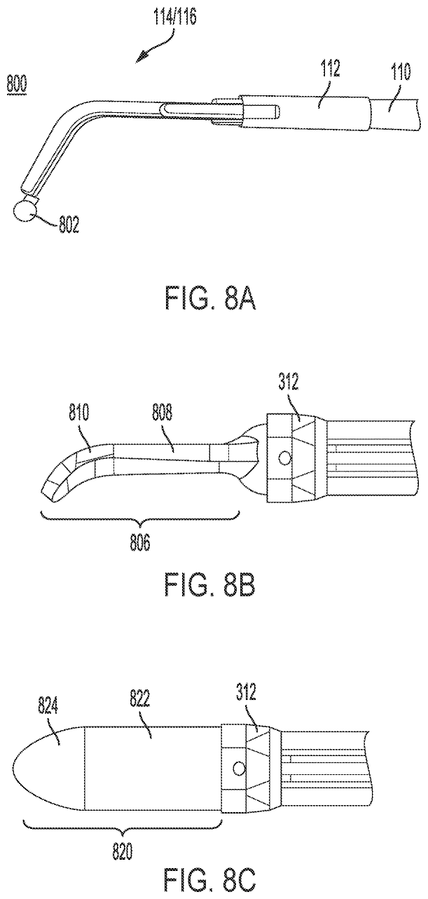

FIGS. 3A and 3B illustrate the support surfaces 206, 210 as members 302, 303 for both jaws (114 and 116, respectively), which support the pad surfaces 310, 311 (corresponding to the pad surfaces 208, 212 in FIG. 2B of jaws 114, 116 respectively). Pad surfaces 310, 311 can be insulating surfaces (e.g., thermally and/or electrically). Further, FIG. 3A shows that the jaw 114 also includes an atraumatic bumper tip receptacle 312, which can itself be configured to receive bumper tips according to a variety of configurations including those exemplarily illustrated in FIGS. 8A-8C discussed further below. Further, FIGS. 3A and 3B show that each jaw 114, 116, includes a respective base 308, 309 corresponding to jaws 114, 116 respectively (e.g., a first surface related to the jaw 114 and a third surface related to the jaw 116 in some examples). This base 308, 309 includes at least the portion of the pad surfaces 310, 311 of respective jaws 114, 116 (e.g., a second surface related to the jaw 114 and a fourth surface related to the jaw 116 in some examples) that faces towards tissue (instead of facing the other jaw or directly away from the other jaw), for example at an angle (e.g., 90 degrees) from the portions of the pad surfaces 310, 311 that face the respective pad surface 310, 311 of the other jaw (e.g., pad surfaces 310, 311 facing each other).

The surface of at least the base 308, 309 can have respective gripping surfaces as will be discussed further below that aid in tissue capture and retention for and during tissue ablation procedures. It can be useful, for example, to assist with tissue capture by providing friction between the respective jaw and target tissue. At least a portion of respective pad surfaces 310, 311 of each jaw 114, 116 can also include similar gripping surfaces, either extending to the gripping surface of the bases 308, 309 of each jaw 114, 116, or stopping before that point (such that there can be a gap between the gripping surfaces on the pad surfaces 310, 311 and the respective bases 308, 309 of each jaw 114, 116). The gripping surfaces on the pad surfaces 310, 311 can respectively promote tissue retention during ablation procedures with the electrosurgical instrument 106 using the electrodes 304, 306 on jaw 114 and electrodes 305, 307 on jaw 116.

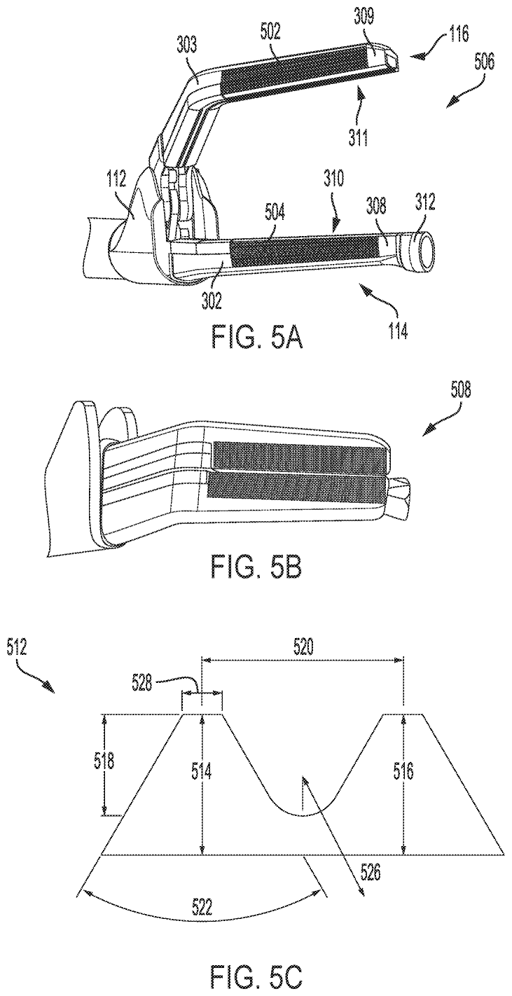

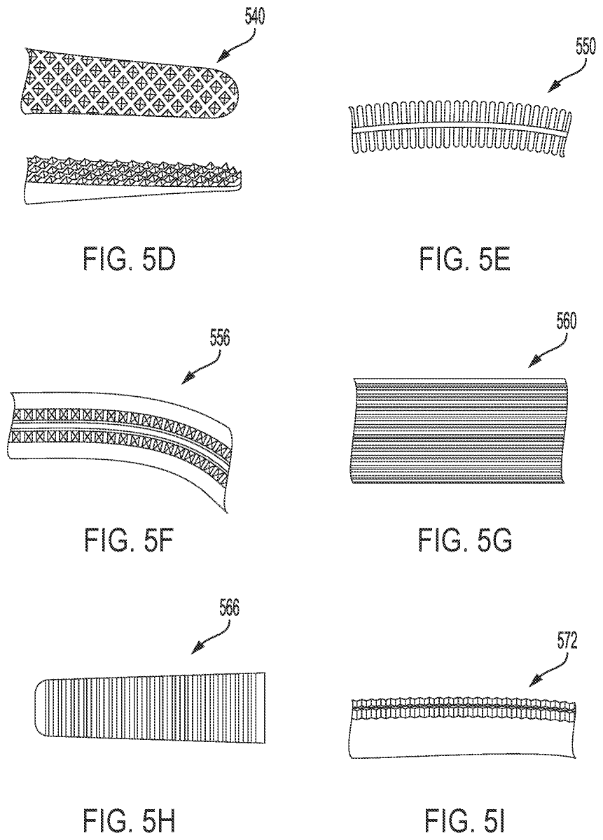

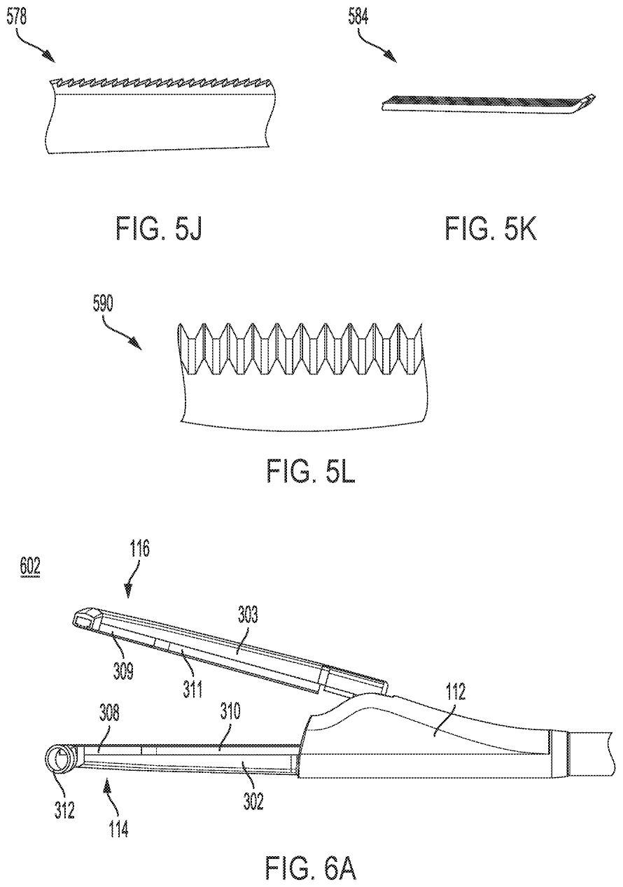

Details of the gripping surfaces are illustrated in more detail with respect to FIGS. 5A-5L. For example, FIG. 5A illustrates an exemplary bottom perspective view of RF tissue ablation jaws 114, 116 of an RF tissue ablation clamp, such as the electrosurgical instrument 106 introduced in FIG. 1, in a first position according to one or more aspects of the present disclosure. As illustrated in FIG. 5A, the bases 308, 309 include gripping surfaces 502, 504—illustrated with gripping surface 502 on the base 309 of jaw 116, and gripping surface 504 on the base 308 of jaw 114. The gripping surfaces 502, 504 can be machined as part of the bases 308, 309, or alternatively can be attached thereto via adhesive, welding, and/or other mechanical coupling. As illustrated in FIG. 5A, the gripping surfaces 502, 504 extend to the edges of their respective jaws 116, 114 that border the pad surfaces 310, 311. FIG. 5A illustrates the jaws 114, 116 in an open configuration. In some exemplary embodiments, the distance between the distal ends of the jaws 114, 116 can be between 0.5 inches and 2 inches (e.g., one inch). This can correspond to a distance that allows both jaws 114, 116 to stay within view of an endoscopic instrument during a surgical procedure, even when in the open position as in FIG. 5A. In response to a user pressing on the plunger 118 (more generally, closure trigger), the jaw 116 begins sweeping towards the jaw 114. Because of the hinge mechanism 112, the jaws 114, 116 sweep together in a manner that provide a generally parallel closure, referring to the jaws 114, 116 being generally parallel to each other (with some variation allowed) as the jaws 114, 116 close on each other. This facilitates an even application of pressure across the length of the respective jaws 114, 116 to the tissue caught and grasped therebetween.

FIG. 5B illustrates the jaws 114, 116 in a closed configuration corresponding to completion of the closing action just described. In operation, as tissue is grasped by the gripping surfaces 502, 504, the jaws 114, 116 will not be fully “closed” as illustrated in FIG. 5B, but rather be at some stage of closure as permitted by the tissue grasped between the jaws 114, 116 and the amount of force applied by the user on the plunger 118 to advance and/or maintain grip on the tissue.

To facilitate the grip on the tissue, according to embodiments of the present disclosure the gripping surfaces 502, 504 can have a design to optimize gripping ability (e.g., to provide friction between jaws 114, 116 and applicable tissue, as well as to promote tissue retention between the electrodes during ablation). For example, the gripping surfaces 502, 504 can have a square or diamond pyramidal configuration, for example a cross-serration pattern. The grasping pattern of jaw 114 and the grasping pattern of jaw 116 can be the same pattern, different patterns, or can have mirrored patterns.

FIG. 5C illustrates an exemplary cross-sectional side view 512 of a gripping surface of an RF tissue ablation jaw, such as either of gripping surfaces 502, 504 of FIGS. 5A-5B according to one or more aspects of the present disclosure. FIG. 5C specifically illustrates the cross-section of two pyramids in the cross-serration pattern from FIGS. 5A-5B, pyramids 514 and 516. The pyramids 514, 516 can have a pitch 520 between them, for example in the range of one hundredth of an inch to five hundredths of an inch (e.g., 26 hundredths of an inch as just one example, which can also be referred to as a medium coarse grain feature). Although illustrated in a two-dimensional configuration, the same pitch can exist between pyramids along multiple axes within the plane they are situated. Further, the pyramids 514, 516 can have a height 518. For example, height 518 can be in the range of five thousandths of an inch to two hundredths of an inch (e.g., 13 hundredths of an inch as just one example).

In some examples, the sides of each pyramid 514, 516 can have a fixed slope until each pyramid's side reaches another pyramid's side. In other examples, the sides of pyramids 514, 516 can have a fixed slope for a first portion of the respective pyramids (e.g., from a top of each pyramid 514, 516 to some region on a side of each pyramid) that transitions into a concave line that curves between pyramids 514, 516 to connect the pyramids together. This exemplary embodiment is illustrated in FIG. 5C with the radius of curvature 526. For example, the radius of curvature can have a value in the range of 3 thousandths to 8 thousandths of an inch (e.g., five thousandths of an inch as just one example).

The slopes of each side of the pyramids 514, 516 can be defined based on an angle of the top of each pyramid 514, 516. For example, each pyramid's top can have an acute angle from which all of the sides extend towards the pyramid's base. This is illustrated as angle 522 in FIG. 5C. The angle 522 can assume a variety of values, for example in the range of 45 to 75 degrees, such as 60 degrees as just one example. In some embodiments, the angle in both dimensions of the gripping surface can be the same, resulting in uniform pyramids 514, 516. In other examples, the angles can be different from each other, meaning that there is a resulting bias in one of the two directions. The direction of this bias can be determined based on a target force profile.

As illustrated, each of the pyramids 514, 516 has a flat surface defining the tip of each pyramid. This flat surface can have a width 518, for example ranging from two thousandths of an inch to ten thousandths of an inch (e.g., 5 thousandths of an inch as just one example). Each pyramid in the configuration of the gripping surfaces 502, 504 (FIGS. 5A, 5B) can have a same approximate width at their tip, i.e. The pyramids can have flat tips as opposed to pointed tips. This can be beneficial for ease of manufacturing (and reduced cost), as well as provide a less sharp gripping surface generally for a better atraumatic result, while also increasing the surface area for gripping with the jaws 114, 116 configured with the pyramidal gripping surfaces 502, 504. In other examples, the pyramids 514, 516 can have tips that reach points, resulting in a sharper grip.

In some examples, the gripping surfaces can extend onto the pad surfaces 310, 311 (FIG. 3A, 3B) as well, such as discussed with respect to FIG. 3A. Thus, the edge on each jaw 114, 116 between the pad surfaces 310, 311 and the gripping surfaces 504, 502 respectively can have a specific geometry as a result of the textured surfaces. Description herein will reference both bases 308, 309 and surfaces 310, 311 with respect to jaws 114, 116, but the description can be generally applicable to either jaw alone or together. For example, with respect to a given jaw 114/116, at the edge of the base 308/309 a pyramid of the gripping surface (e.g., pyramid 516 in FIG. 5C) can terminate as a half-pyramid at the edge adjacent to the pad surface 310/311. Likewise, at the edge of the pad surface 310/311 a pyramid of the gripping surface (e.g., pyramid 514 in FIG. 5C) can terminate as another half-pyramid at the edge adjacent to the base 308/309 (of respective jaws 114/116).

Thus, both sides can terminate with a half-pyramid, which when viewed together can be viewed as a square feature orientation in the transition from the base 308/309 to the pad surface 310/311, and likewise when transitioning from the pad surface 310/311 to the base 308/309. Together, the griping surfaces 502, 504 can thus extend on a first surface corresponding to at least part of the pad surface 310/311, on a second surface corresponding to at least part of the base 308/309, and further on a third surface corresponding to the boundary between the two (pad surface 310/311 and base 308/309). In other embodiments, the third surface can be a wider bevel that transitions between the pad surface 310/311 and base 308/309, and can include a gripping surface likewise configured with pyramids or other textures to promote gripping and retention of tissue during procedures such as those provided herein.

The gripping surfaces 502, 504 can have alternative configurations to the cross-serration pattern discussed above with respect to FIG. 5C. For example, FIG. 5D illustrates an example with an alternative cross-serration pattern where the pyramids have a diamond shape within the plane of the gripping surface, as opposed to a square configuration within the plane of the gripping surface. As another example, FIG. 5E illustrates an example with teeth configured similar to that used with anastomosis clamps. As another example, FIG. 5F illustrates an example with teeth configured similar to that used with tangential occlusion clamps. As another example, FIG. 5G illustrates an example with teeth configured similar to that used with bronchus clamps. As yet another example, FIG. 5H illustrates an example with teeth configured similar to that used with thoracic artery forceps. As another example, FIG. 5I illustrates an example with teeth configured as Cooley toothing. As another example, FIG. 5J illustrates an example with teeth configured similar to that used with DeBakey toothing. FIG. 5K illustrates yet another example with teeth configured with longitudinal serrations, and FIG. 5L illustrates an example with teeth configured with horizontal serrations. Additionally, the teeth can be cuboidially-shaped. Each of the plurality of cuboidially-shaped teeth can culminate in a pointed, flat, chamfered or filleted surface before reaching a pyramid tip.

In addition to different teeth configurations, the gripping surfaces of embodiments of the present disclosure can have other aids to facilitate grasping tissue and/or retaining tissue by jaws 114, 116. For example, suction can be integrated along at least part of the length of one or both jaws 114, 116 to aid in gripping tissue. The suction integration can take the form of one long, narrow outlet extending along the part of the length of the jaws, or the form of multiple small suction ports along a line extending along the part of the length of the jaws. The outlet or suction ports can be connected to a vacuum source that can in some embodiments be integrated with the RF generator 102 (FIG. 1), or in other embodiments part of a separate unit. The connection can extend via a lumen within the elongated longitudinal shaft 110 and connect either via port 120 or another port not shown to such vacuum source.

As another example, one or both jaws can include one or more cryogenic points along their lengths. These can provide cryogenic energy from the cryogenic points that can produce “tack” and subsequent tissue grip. The cryogenic energy can be provided via a cryogenic source, providing cryofluid to the points of contact along the lengths of the jaws and can generate surface temperatures below −40 degrees Celsius. Devices and systems implementing cryogenic energy are described in U.S. Pat. No. 8,915,908, which is incorporated herein by reference.

As another example, instead of machining the gripping surfaces 502, 504 with patterns to facilitate the gripping ability, embodiments can apply clip-on tissue grip aids. These can be, for example, interchangeable inserts, soft inserts, soft polymer with Velcro loops, serrated soft polymer, latis inserts, and/or rigid inserts to name a few examples. Other insulator materials and/or textures can alternatively be applied for or with the gripping surfaces 502, 504 to facilitate both the grasping of tissue (e.g., as at least one jaw sweeps along tissue during a closing motion) as well as retention of grasped tissue during an ablation procedure by the electrodes 304/305, 306/307 of both jaws 114, 116. Additionally, the gripping surfaces 502, 504 can also be achieved in various alternative methods such as cutting (e.g., etching), knurling, coining, stamping patterns into the metal of the jaws 114, 116, or applying an abrasive coating to the jaw.

Returning to discussion of FIG. 3A, as illustrated the electrodes 305, 307 extend from a proximal end 324 of jaw 116 (i.e., proximal to the hinge mechanism 112) to a distal end 328 of jaw 116 (i.e., the tip of the jaw 116). While not shown from the perspective of FIG. 3A, the jaw 114 can have electrodes 304, 306 similarly extending from the proximal end 322 of jaw 114 to hinge mechanism 112 to the distal end 326 of the jaw 114, stopping for example at the atraumatic bumper tip receptacle 312. Thus, together the jaws can include two pairs of opposed electrodes (304, 306 and 305, 307). Alternatively, the electrodes 304/305, 306/307 can start at a location on the proximal portion of the jaws 114, 116 that is not adjacent to the hinge mechanism 112 (i.e., a distance away from the hinge mechanism 112 but before the bend in each jaw). The electrodes 304/305, 306/307 can further be set within respective insulators that extend along the length of the jaws 114, 116 from the proximal ends to the hinge mechanism 112 to the distal ends at the tips of the jaws 114, 116 (where the electrodes extend in this manner—more generally, the electrodes 304/305, 306/307 can be set within respective insulators for the full amount they extend along each respective jaw 114, 116).

For example, the electrodes 304, 306 on the first jaw 114 (FIG. 3B) can be centered laterally on the pad surface 310 about the medial plane of the first jaw 114 and can be spaced apart from each other in a range of from about 0.7 mm to 4 mm Other ranges are possible as well; the distance can be a factor of the thickness of the tissue to be ablated. Other factors influencing the distance between the electrodes 304, 306 on the first jaw 114 can include the type of tissue (e.g., cardiac, skeletal muscle or smooth muscle, such as the small bowel or uterus wall), the frequency of energy source switching, the desired duration of ablation, and the desired width of ablation. As an example, if the tissue to be ablated is about 5 mm thick (uncompressed), which is typical of cardiac tissue, the preferred electrode spacing can be approximately 1.0 mm. As the spacing of the electrodes is increased, it is preferred that the insulator surface be convex so that the desired increased pressure on the tissue between the electrodes is achieved. With two pairs of opposed electrodes, the crown radius of the insulator of an embodiment can be about 4.5 mm, and its face width about 5 mm. These dimensions are illustrative only.

The second jaw 116 can be configured similarly to the first jaw 114 with electrodes 305, 307 opposite to electrodes 304, 306 on first jaw 114. The electrodes 304/305, 306/307 on each of jaws 114, 116 can be the same size and shape to each other. The electrodes 304/305, 306/307 on each jaw 114, 116 can, for example, be formed with a beryllium-copper base with a nickel/gold plating covering exposed surfaces. The electrodes 304/305, 306/307 of each jaw 114, 116 can have a preferred electrode width of about 0.012 inches (as an example) and can extend from the surface of the insulator over the length of the ablation surface from about 0.000 mm to about 0.15 mm (as an example—other widths and projections can also be used).

The electrodes 304/305, 306/307 of respective jaws 114, 116 can form parallel dual electrode arrays that can be pulsed. That is, electrodes 304/305 on jaws 114, 116 can define one ablation energy source in the form of one pair of opposed electrodes having opposite polarity and electrodes 306/307 on jaws 114, 116 can define another energy source in the form of a second pair of opposed electrodes that have opposite polarity. In some embodiments, the electrodes on each jaw can be of the same polarity, while in other embodiments the electrodes on each jaw can be of the same or opposite polarity. When energized by the RF generator, electrical current flows between the electrodes, creating an energy field in the form of current flow or flux between the electrodes.

As illustrated in FIG. 3C, insulator components 314, 316 can be detachable from the jaws 314, 316 as desired. In some variations, the insulator components can be configured with clips and snap fit receptacles on the jaws 114, 116. Clips and snap fit receptacles can eliminate the need for bonding between the jaw and insulator components. The insulator components 314, 316 can comprise textured surfaces 318 and can be incorporated as part of the insulator (for example, via 3D printing for prototyping and injection molding for production). This can allow for increased flexibility in terms of pattern and manufacturability.

In some examples, the two energy sources or pairs of opposing electrodes can be pulsed by energizing the first electrode pair for a timed duration, turning the first electrode pair off, energizing the second electrode pair for another, preferably the same, timed duration, turning the second electrode pair off, and repeating the pulsing cycle (alternately energizing and de-energizing the first electrode pair and the second electrode pair), thus creating an area of overlapping current flux. The overlapping area of current flux is thus subjected to a substantially continuous current flow, while the other tissue, including tissue at the electrode interface, experiences only intermittent current flow. As a result, a zone of primary tissue heating is created in the area of overlap, which is spaced from the area of highest current density.

The cycling of the electrodes can be repeated until the ablation of the tissue is transmural. Thus, to achieve a transmural ablation line, the opposing pairs of electrodes can be cycled in a series of on-off first pair, on-off second pair cycles until ablation is complete. Transmural ablation can be achieved when the temperature of the tissue reaches approximately 50° C. Thus, one or more temperature sensors can further be associated with the jaws to provide an indication of the progress of ablation. The temperature sensor(s) can be located on one or both jaws between the electrodes forming part of each opposed pair or on the jaws outside of the electrodes (e.g., on the pad surfaces 310/311 or on the respective bases 308/309 of each jaw 114, 116) to detect thermal spread, which is the lateral spread of heat from the area or zone of ablation into tissues outside the zone.

Returning to FIG. 4B, each of the electrodes 304/305, 306/307 (FIGS. 3A, 3B) of the respective jaws 114, 116 can be connected to the RF generator 102 (FIG. 1) via leads that can extend through the hinge mechanism 112 and through the center of the elongated longitudinal shaft 110, either outside the inner shaft 214 (e.g., between an inner surface of the elongated longitudinal shaft 110 and an outer surface of the inner shaft 214 in lumen 416 (FIG. 4D)) or in the inner shaft 214 (e.g., in lumen 414 (FIG. 4D)).

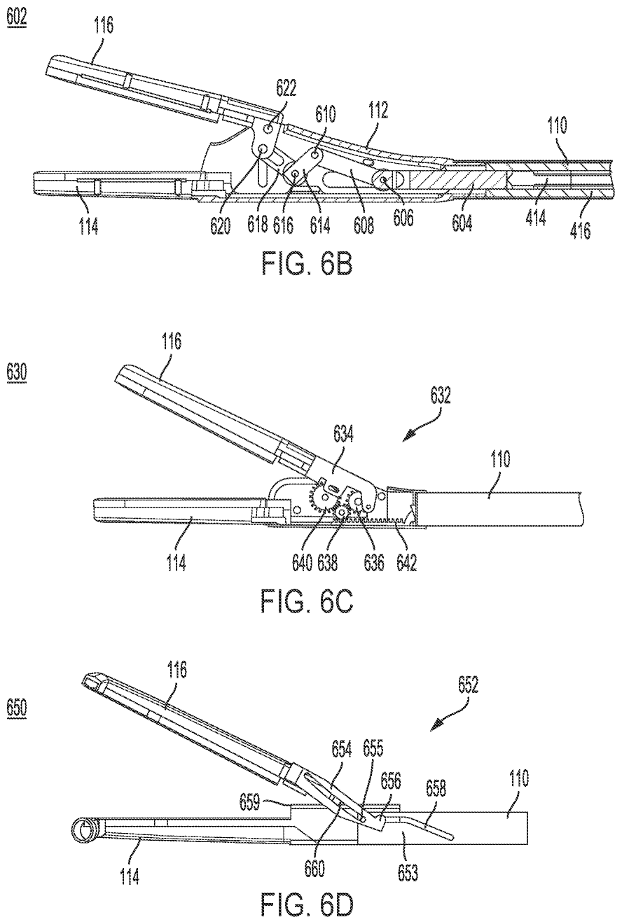

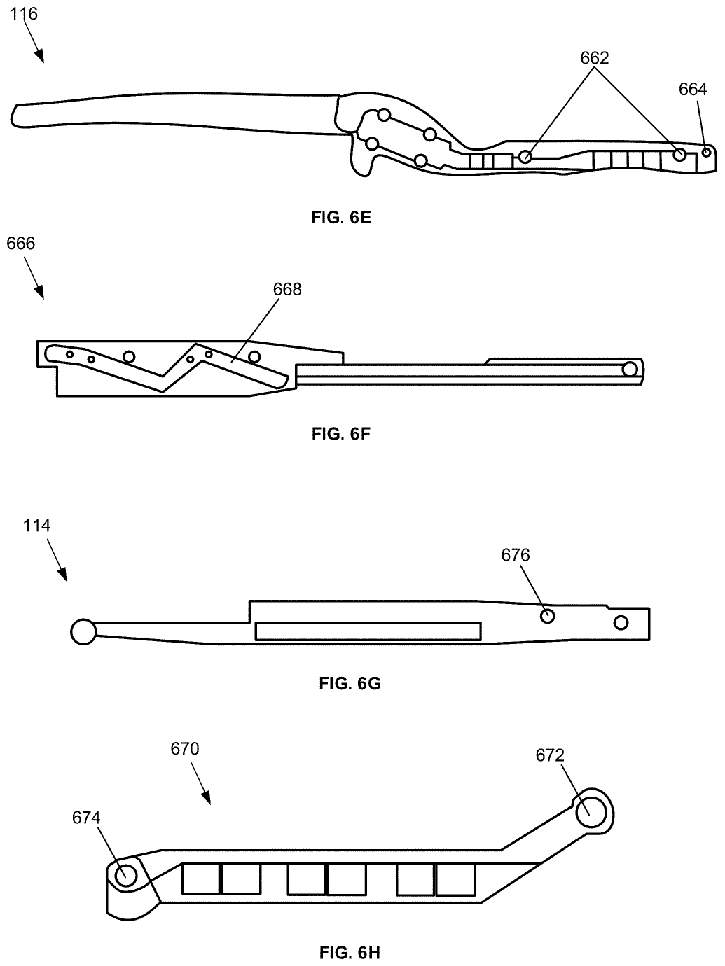

Focusing in particular on the hinge mechanism 112, according to embodiments of the present disclosure the hinge mechanism can have a height that is sufficiently small to fit within a trocar incision, for example a 12 mm trocar incision. Looking, as an example, at FIG. 6A, an exemplary front view of RF tissue ablation jaws with a hinge of an RF tissue ablation clamp according to one or more aspects of the present disclosure is illustrated. FIG. 6A in particular shows the height of the hinge mechanism 112 along a vertical axis of the illustration. The height of the hinge mechanism 112 can be around 20 mm or less in order to fit within such a trocar incision. The hinge mechanism 112 can control action with tactile feedback, such as with 1:1 force and motion, to maximize user control. The hinge mechanism 112 can have a fixed angle of about 30 degrees.

Turning now to FIG. 6B, an exemplary cross-sectional view 602 of the hinge mechanism 112 and jaws 114, 116 is illustrated according to one or more aspects of the present disclosure. As shown, the distal end of the elongated longitudinal shaft 110 connects to a proximal end of the hinge mechanism 112.

Within the hinge mechanism 112, an inner shaft 214 can connect via linkage 604 to a first linking arm 608 via coupling 606. The coupling 606 can be, for example, one or more pins through respective ends of linkage 604 and first linking arm 608. The first linking arm 608 can in turn connect via coupling 610 to second linking arm 614, which in turn can connect to third linking arm 618 via coupling 616. The third linking arm 618 can connect to a base portion 622 of the jaw 116. The jaw 114 can be fixed with the hinge mechanism 112 such that it does not move relative to the jaw 116, but instead serves as an anchor to the movement of jaw 116 to provide a level of parallel closure from a beginning of a stroke to an end of the stroke (e.g., corresponding to full closure against the jaw 114 or gripping of tissue between jaws 114 and 116). Moreover, the parallel closure can provide a relatively consistent pressure profile during the stroke of the jaw 116 in closure.

Upon actuation of the plunger 118, the actuation can be transferred via one or more linking cables or electrical signals to the linkage 604. The linking arms and couplings can be placed to promote a slim profile (e.g., height) to facilitate the desired fit in a small trocar incision. The configuration illustrated in FIG. 6B can be a custom configuration of a bell-crank mechanism.

As an alternative, FIG. 6C illustrates an exemplary cross-sectional view 630 of a hinge mechanism 632 (e.g., an alternative implementation of hinge mechanism 112) and jaws 114, 116 according to one or more aspects of the present disclosure. Instead of using a bell-crank mechanism, FIG. 6C illustrates an alternative that implements a rack-and-pinion approach. Within the hinge mechanism 632, an inner shaft 214 can connect via rack 642 (i.e., a linear actuator/gear) to a pinion 638 (e.g., a first circular gear). The pinion 638 can have multiple teeth to interact with gears 636 and 640. When the plunger 118 is actuated, that can be transferred via one or more linking cables or electrical signals to the rack 642. When the rack 642, the linear motion is translated to rotational motion via the pinion 638. The motion of the pinion 638 further causes the gears 636 and 640 rotate, causing the jaw 116 to move in response to connection via the proximal portion 634 of the jaw 116.

As another alternative, FIG. 6D illustrates an exemplary cross-sectional view 650 of a hinge mechanism 652 (e.g., another alternative implementation of hinge mechanism 112) and jaws 114, 116 according to one or more aspects of the present disclosure. Instead of using a bell-crank or rack-and-pinion approach, FIG. 6D illustrates an implementation of a dual-cam approach. Within the hinge mechanism 652, the distal end of the elongated longitudinal shaft 110 can attach to a slider 653. The slider 653 can include a first cam cutout 658. Alternatively, embodiments can provide for the inner shaft 214 (FIG. 2B, e.g., a drive bar) to include the slider 653 (and corresponding first cam cutout 658) and perform the operations described below with respect to the elongated longitudinal shaft 110. For simplicity of discussion below, aspects with be described with respect to the elongated longitudinal shaft 110.

The first cam cutout 658 can extend along an angled path along the slider 653, for example corresponding to a range from one side of the elongated longitudinal shaft 110 towards the other side of the elongated longitudinal shaft 110 (e.g., within a housing of the device generally), providing a path for a lever 654 of the jaw 116 to travel for actuating the jaw 116. The hinge mechanism 652 can further include a pivot 656 attached or part of the lever 654 (e.g., a pivot pin through the first cam cutout 658). The hinge mechanism 652 can also include a fulcrum 655 attached to or part of the slider 653 (e.g., that is through the second cam cutout 660). The lever 654 can travel along the first cam cutout 658 via pivot 656. Further, the slider 652 can travel along the second cam cutout 660 via fulcrum 655. The distal end of the elongated longitudinal shaft 110 can extend into a receiving housing 659. The first jaw 114 can be fixed to the receiving housing 659, such as at a distal end thereof. The lever 654 can include a second cam cutout 660, which can provide a point at which to connect the lever 654 to the receiving housing 659. The second jaw 116 can be coupled near the distal end of the receiving housing 659 via the lever 654.

In operation, activation of the plunger 118 can transfer the actuation to the receiving housing 659 by causing the elongated longitudinal shaft 110 to move distally towards the receiving housing 659 (or, in other embodiments, a connection within the longitudinal shaft 110; for purposes of discussion here, reference will be made simply to the longitudinal shaft 110 moving, while it will be recognized that the connection could instead move while the shaft 110 remains stationary). When the plunger 118 is pressed, the fulcrum 655 attached to the slider 653 can move distally, causing the jaw 116 to rotate about the pivot 656 due to the second cam cutout 660 in the lever 654 associated with jaw 116. The input plunger/slider stroke can be, in some examples, approximately 1.0 inch which can provide ideal user ergonomics.

As the plunger 118 is pressed, the jaw 116 can rotate to a position parallel to the orientation of the jaw 114. This can occur, for example, within the first approximately 30 to 40 percent range of the plunger stroke (e.g., 0.3 to 0.4 inches in one example) to complete the rotational portion of the jaw 116's movement. As the fulcrum 655 moves away from the pivot 656 (e.g., a pivot pin), mechanical advantage within the hinge mechanism 652 can increase. Because of this, the jaw 116 can apply more force as the jaws 114, 116 close to compress target tissue. The rotational movement portion of the hinge mechanism 652 can account for a significant amount of the total closure of the jaws 114, 116, e.g., between 60 and 90 percent (e.g., about 80% in one example). A remaining amount of the total closure (referring to the jaw 116's movement during closure) can be linear in nature (e.g., in some examples around 0.2 inches or 5 mm). This generally linear portion of closure can provide higher force than the rotational portion of closure of hinge mechanism 652 (e.g., significantly higher force) to compress the tissue.

For the linear portion of travel (relating to closure), as the first cam cutout 658 in the slider 653 (which can function by constraining the pivot 656) moves distally (from the plunger 118 continuing to be pressed), the movement can force the pivot 656 down at approximately the same rate as the fulcrum 655 pushes down the jaw 116 (down, as in towards the jaw 114). This can result in linear motion of the jaw 116 relative to the lower jaw 114 (e.g., a linear closing motion of the jaw 116 towards the jaw 114). In some examples, a stroke of the slider 653 can be in the range of 0.6 to 0.7 inches (as just one example). With that range, jaw closure (movement of jaw 116) can result in approximately 0.2 inches of jaw closure, with a potential mechanical advantage of more than 3:1. In addition, at the end of travel of the slider 653, the fulcrum 655 can move approximately 1 to 2 inches (e.g., 1.2 inches) away from the pivot 656. This movement can minimize the stresses on the components so they can fit in minimal space.

The clamping force can play a key role in preventing a complication at times called “Steam Popping.” A steam pop can be when the fluid being ablated with RF energy gets hot enough to vaporize and “pop.” Such popping can cause trauma and bleeding to the ablated area. A target pressure for the clamp can be 16-psi over an estimated area of between 0.3 and 0.5 square inches (such as 0.43 as just one example) so the force on the clamp can be nearly 7.0 pounds. The pressure under the clamp (e.g., between jaws 114, 116) can squeeze out excess moisture, and can also increase the local pressure (which significantly increases the vaporization temperature). For reference, water at normal pressure transitions to the vapor state (boils) at 100° C. At a pressure of 16 psi, however, the vaporization temperature is higher (e.g., around 22° C.). This increase can provide a good margin of safety factor from steam popping complications.

Since the electrosurgical instrument 106 can be, in some examples, intended to be inserted through the intercostal space between the ribs, it is useful to minimize the outside dimensions in order to reduce the patient's pain. Since the jaws 114, 116 can be angled and/or curved, they might not be inserted through a trocar. However, the jaws 114, 116 and hinge mechanism 652 can fit in the incision left by the trocar after the trocar is removed. The intercostal space between the ribs varies between patients but can generally be less than 0.5 inches. Nerves run on the bottom side of the ribs, so it can be desirable to minimize interaction with them in order to reduce the patient's post-procedural pain. Keeping the outside dimensions less than 0.5 inches can greatly reduce nerve interaction and injury to minimize post-procedural pain.

As a result, aspects of FIG. 6D can result in an upper jaw with a rotational motion followed by a true linear clamping motion. The rotational motion can facilitate presenting frictional features on the distal side of the jaw 116 to engage and grab target tissue to pull it into a clamping area. The linear clamping portion of the stroke can provide linear motion so that the distal and proximal electrodes are parallel. Further, the output clamping force can be non-linear compared to the input force. For example, the beginning of the rotational part of the clamping motion can have less force and more motion per plunger 118 movement, while a linear portion of the stroke can have a mechanical advantage for increased clamping pressure to minimize a potential for steam pops. For example, an RF ablation clamp can result with a non-linear output force compared to input force where less than 40 percent (in some examples) can account for around 80 percent of the output closing stroke, and a remaining portion of the input stroke can account for around 20 percent of the output stroke in order to provide a mechanical advantage to obtain higher clamping pressures to mitigate steam popping injuries (among other things).

Access can be gained through the intercostal space between ribs, for example, so as with other embodiments an outside dimension of the clamp configuration can be kept as small as possible. The cam design of FIG. 6D can take advantage of geometry with a moveable fulcrum to reduce stresses on the components to minimize the outside dimensions.

The device can be various positions during opening of jaws 114, 116. The jaws 114, 116 of the device can open as wide as they can to accommodate loading tissue. The end effector of the device can be sized for access into and out of the ribs. In a closed position, the overall height of the device can be 12.7 mm and the width can be 10 mm. The hinge mechanism 112 can achieve an opening between the jaws with a true parallel travel over a range of 0-14 mm, for example 5.5 mm. When the clamp latches with the desired pressure profile, the tissue can compress to approximately 40% of its uncompressed height, yielding a total compressed thickness of 5.3 mm. The jaw tip to jaw tip distance in the open position can be 25.6 mm.

In another variation, FIGS. 6E to 6H illustrate an alternative mechanism for opening and closing of the first and second jaws 114, 116 via a sliding cam 666. The sliding cam 666 can travel axially inside the fixed jaw 114 and shaft 110. The moving jaw 116 can sit inside the sliding cam 666 attached via bearings 662. A link 670 can connect the moving jaw 116 and fixed jaw 114 attached via connections 672, 674. The moving jaw 116 can have bearings 662 mounted at a more proximal and more distal portion of the moving jaw 116. The bearings 662 can move inside track 668 in the sliding cam 666 which can cause the moving jaw 116 to follow the desired motion. Axial motion of the sliding cam 666 can cause the moving jaw 116 to open and close as desired. The moving jaw 116 can be attached to the fixed jaw 114 via a link 670. The moving jaw 116 can have two locations for mounting bearings 662, and a connection 664 to the link 670.

As shown in FIG. 6F, the sliding cam 666 can have with track 668 for the bearings 662 to move within on the moving jaw 116. The track 668 can be shaped in a zig-zag pattern along the longitudinal axis of the sliding cam 666. The sliding cam 666 can be slightly less wide than the fixed jaw 114 to permit movement within the fixed jaw 114.

As shown in FIG. 6G, the fixed jaw 114 can have a pin 676 for a connection to the link 670 of the moving jaw 116. The pin 676 can be located towards a proximal end of the fixed jaw 114. The pin 676 can be placed within the connection of the link 670 to facilitate movement of the jaws 114, 116 while the sliding cam 666 is moved axially.

As shown in FIG. 6H, the link can have a connection to fixed jaw 114 and the moving jaw 116. The link 670 can prevent axial motion of the moving jaw 116 relative to the fixed jaw 114. The link 670 can be placed at the proximal end of the moving jaw 116. The link 670 can have a connection 672 to the pin 676 of the fixed jaw 114 located at a proximal end of the link 670. The link 670 can have a connection 674 to the moving jaw 116 at a located at a distal end of the link 670.

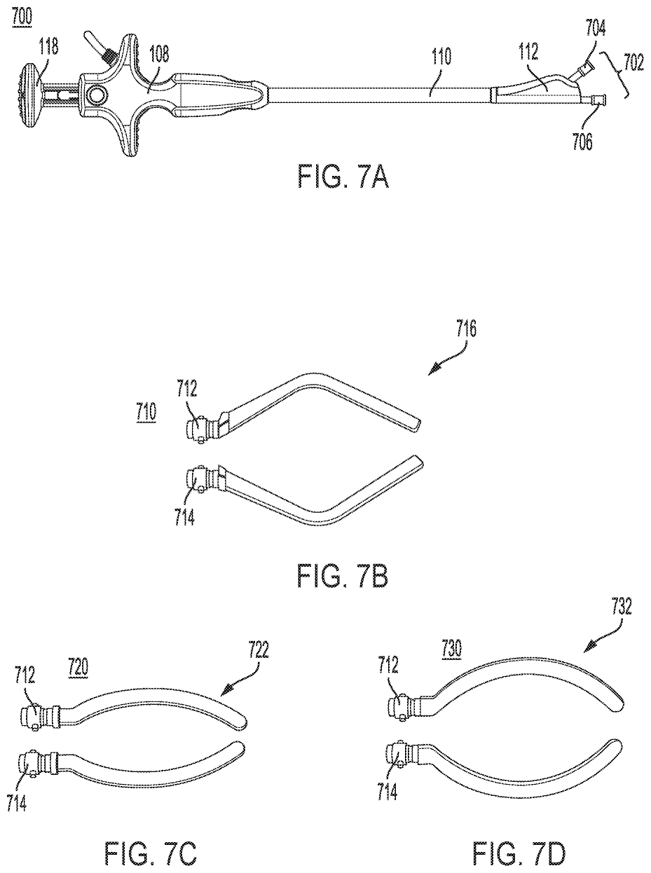

Aspects of the present disclosure have been discussed above with respect to FIGS. 1-6D as having jaws 114, 116 that can be fixedly attached to one or more linkages associated with the hinge mechanism 112. In alternative embodiments, however, the jaws can instead be removable and/or interchangeable with different jaw configurations. For example, FIG. 7A illustrates an exemplary top plan view of an RF tissue ablation clamp 700 with interchangeable jaws according to one or more aspects of the present disclosure. Jaws can be removed by pressing ball bearings out of detents on hubs located on the shaft 110.

Many aspects of the clamp 700 are similar to those discussed already, for example the handle 108, elongated longitudinal shaft 110, hinge mechanism 112, and plunger 118. However, instead of fixed jaws 114, 116, the clamp 700 has end effector 702 with two receiving couplings for respective first and second jaws. For example, where fixed jaw 114 would have been, end effector 702 can have a receiving coupling 706 for a first interchangeable and/or replaceable jaw (for simplicity of discussion, referred to below as simply interchangeable jaws but which is still applicable to replaceable jaws as well). This can correspond to the jaw that does not move relative to the other jaw (thus serving as an anchor therefor). Further, where movable jaw 116 would have been, end effector 702 can have a receiving coupling 704 for a second interchangeable jaw. Each receiving coupling 704, 706 can include mechanisms to secure jaw ends upon insertion, whether that be by threading, ball joints, or other approaches. Further, each receiving coupling 704, 706 can include electrical connections to connect to electrodes on the interchangeable jaws. By providing for interchangeable jaws, the user can be provided with more flexibility without needing to open a separate clamp with a different target jaw curvature.

For example, FIGS. 7B-7D illustrate exemplary views of interchangeable jaw configurations for an RF tissue ablation clamp, such as the clamp 700 of FIG. 7A, according to one or more aspects of the present disclosure. For example, in FIG. 7B, a first set of interchangeable jaws 710. The first set of interchangeable jaws 710 can have a configuration similar to that of jaws 114, 116 of FIGS. 1-6D above, e.g. With proximal portions that are generally parallel to a plane along which an elongated longitudinal shaft of the clamp 700 extends. The jaws 710 can further have distal portions that are set at an angle to the plane in which the proximal portions are situated, such as an angle in a range of 110 degrees to 130 degrees (such as 120 degrees).

The jaws 710 can include respective end connectors 712, 714 that include respective mating mechanisms compatible with the receiving couplings 704, 706. In some embodiments, the end connectors 712, 714 can be generic so that either jaw can be attached to either of the receiving couplings 704, 706. In some other embodiments, the end connectors 712, 714 can have specific coupling configurations that are unique to respective ones of the receiving couplings 704, 706 such that they will only fully couple with their intended one of the receiving couplings. In such situations, the receiving couplings 704, 706 and/or the end connectors 712, 714 can include respective markings to designate their matches. Upon insertion of the end connectors 712, 714 into the appropriate receiving couplings 704, 706 (if applicable), electrical connections can be closed so that electrodes on the jaws 710 can receive energy upon command from the RF generator 102 (FIG. 1).

FIG. 7C illustrates a second set of interchangeable jaws 720. In contrast to the jaw configuration in FIG. 7B, the second set of interchangeable jaws 720 can have a consistent radius of curvature (as opposed to two linear portions set together at an angle as in FIG. 7B). The radius of curvature can result in the second set of interchangeable jaws 720 having a first concavity. Like the jaws 710 in FIG. 7B, the jaws 720 in FIG. 7C can include respective end connectors 712, 714 for coupling with the receiving couplings 704, 706 and operate as discussed above. FIG. 7D illustrates a third set of interchangeable jaws 730. Jaws 730 can also have a consistent radius of curvature, like jaws 720 of FIG. 7C, albeit with a second concavity that is greater than that illustrated in FIG. 7C.

FIGS. 8A-8C illustrate exemplary side views of alternative atraumatic bumper tip configurations for an RF tissue ablation clamp according to one or more aspects of the present disclosure. For example, FIGS. 8A-8C can illustrate examples of atraumatic bumper tips that can fit into the atraumatic bumper tip receptacle 312 introduced with respect to FIG. 3A above.

FIG. 8A illustrates the atraumatic bumper tip configuration 800 as using a ball tip 802 inserted into the atraumatic bumper tip receptacle 312 (FIG. 3A). The ball tip 802 can be inserted into the receptacle 312 in a releasable way, or can alternatively be permanently attached. The ball tip 802 can be used as a simple bumper type tip, to reduce instances of the end of jaw 114 catching on surrounding tissue during a procedure.

The ball tip 802 can be formed of a variety of materials, for example some grade of polyester or other plastic that provides some flexibility and impact energy absorption. The ball tip 802 can have a base with a cross-sectional size similar to that of the distal end of the jaw 114, and can for example be slightly smaller to facilitate insertion/connection into the atraumatic bumper tip receptacle 312. The ball tip 802 can then expand in radius for the ball portion extending beyond the jaws 114, 116. Moreover, the ball tip 802 can be provided in a variety of colors. In some examples, the ball tip 802 can be a blue color, a red color, or a white color in order to increase visibility of the ball tip 802 in contrast with surrounding tissues.

FIG. 8B illustrates an alternative bumper tip configuration with tip 806, also referred to as a ski tip bumper tip 806 in view of its dimensions and appearance. The tip 806 can include a proximal portion 808 that is generally flat with a given thickness along its length. This can connect to the receptacle 312 by a similar approach as that described with respect to FIG. 8A above. The tip 806 can further include a distal portion 810 connected to the proximal portion 808. The distal portion 810 can remain generally flat with the same thickness as the proximal portion, or can vary in thickness to either achieve a broader tip (greater thickness) or narrower tip (less thickness).

While the proximal portion 808 can lie in a plane generally in alignment with the plane in which the distal portion of the jaw 114 rests (which is, itself, offset from a plane in which the proximal portion of the jaw 114 rests), the distal portion 810 can be offset from that plane. The proximal and distal portions 808, 810 can connect to each other via a curved portion therebetween. The tip 806 can be formed of a material as discussed above with respect to FIG. 8A. The tip 806 can be used to scoop around internal body parts during a procedure, for example around the inferior vena cava during a heart procedure. The ski tip design can also make it easier to visualize the tip by a user. Moreover, the ski tip design of tip 806 can also allow an endoscopic grasper to grasp it to assist with pulling the rest of the clamp into a target location.

FIG. 8C illustrates an alternative bumper tip configuration with tip 820, also referred to as a bullet tip in view of its dimensions and appearance. The tip 820 can include a proximal portion 822 and a distal portion 824, but unlike FIG. 8B where both portions are flat with a given thickness(es), the proximal portion 822 has a given radius along its length, instead of being flat. The distal portion 824 can likewise have a given radius that begins to get smaller in a conical formation towards the distal tip of the distal portion 824. The tip 820 can be formed of a material as discussed above with respect to FIG. 8A. For example, the tip 820 can be flexible enough that it can deflect off of surrounding structures as well as allow an endoscopic grasper to grasp it to assist in pulling the clamp into place in a target location.

As noted previously, the jaws of an electrosurgical instrument 106 can be inserted through a small incision into a patient. That incision can be, for example, a trocar incision. During a procedure, such as a closed-chest minimally invasive procedure, the body cavity around a tissue and/or organ of interest can be pressurized by a gas, such as carbon dioxide, to provide working space and visualization for the procedure within the cavity (such as a thoracic cavity). However, electrosurgical tools such as the electrosurgical instrument 106 might not fit through standard thoracic trocars (as an example of ports used for access during minimally invasive surgery), due to the size of the electrosurgical instrument 106 and in particular the angled/curved geometry of the jaws.

Normally, to insert an electrosurgical instrument 106 into the body cavity (such as the chest) during a procedure, the user can remove a port (i.e., a trocar) from the chest and insert at least the jaws directly through the skin incision. However, after removal of the trocar, the gas is prone to flowing out around the shaft of the electrosurgical instrument 106 through the skin incision. While this can be countered with a stitch of part of the incision, such as a purse string stitch, to tighten the incision down around the shaft of the electrosurgical instrument 106, this is sub-optimal due to the extra time and manual effort necessary. Others might use a flexible port (trocar) or gel point system to address the issue. These likewise require extra time and effort.

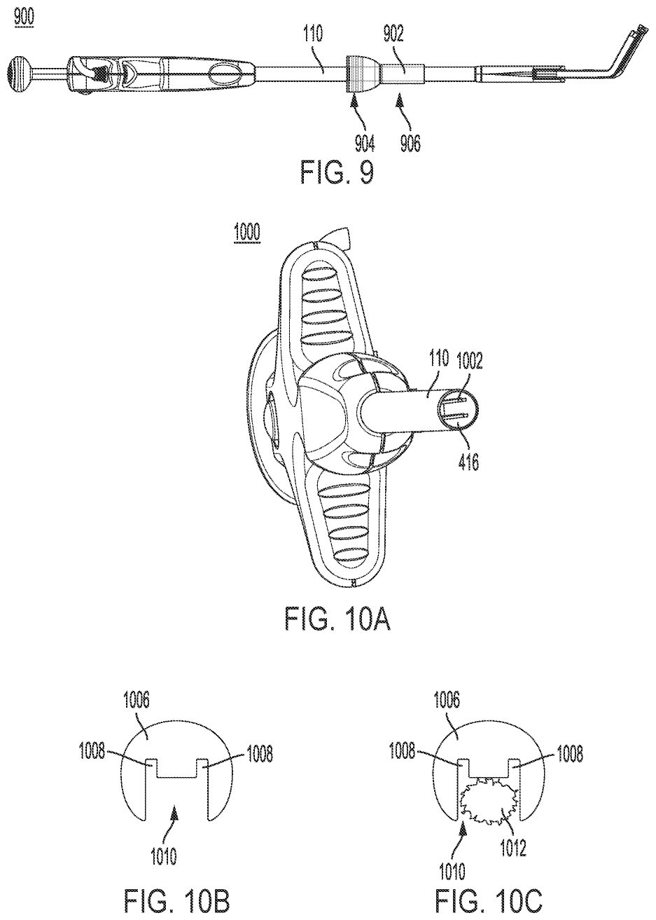

Instead, according to embodiments of the present disclosure, a trocar can be incorporated onto a shaft of an electrosurgical instrument 106. For example, FIG. 9 illustrates an exemplary side view 900 of an RF tissue ablation clamp, such as the distal end of electrosurgical instrument 106 introduced in FIGS. 1-4E, with an incorporated trocar according to one or more aspects of the present disclosure.

As illustrated, a trocar 902 can be incorporated onto the elongated longitudinal shaft 110. The trocar 902 can be incorporated onto the elongated longitudinal shaft 110 in a manner that allows the trocar 902 to slide along the length of the elongated longitudinal shaft 110. For example, the section at the proximal end 904 of the trocar 902 can include a hole cut within the trocar 902 to allow the elongated longitudinal shaft 110 through. The hole can include a ring attached to the edges of the hole made of a flexible material, such as a polymer, that allows movement (e.g., sliding of the trocar 902 along the elongated longitudinal shaft 110) while also preventing some amount of gas (or nearly all) from passing through. The distal end 906 can be similarly configured to allow passage of the elongated longitudinal shaft 110 while preventing some or all of gas from passage.

In use, a user can remove an original trocar from the skin and insert the head of the clamp (e.g., including both jaws 114 and 116, hinge mechanism 112, and/or some length of the elongated longitudinal shaft 110) into the cavity through the incision. The user can then slide the included trocar 902 down along the elongated longitudinal shaft 110 down into the incision instead of relying on some other mechanism, such as a stitch, to impede the release of gas from the insufflated cavity.

In addition to preventing the release of gas from the incision, it can be desirable to also mitigate or stop the release of gas through any device inserted into the cavity, such as where the device is not sealed internally and might provide another avenue for gas release external to the subject. According to embodiments of the present disclosure, therefore, internal components of the electrosurgical instrument 106 can further include elements to impede or wholly seal the inside of the electrosurgical instrument 106.

FIG. 10A illustrates an exemplary cross-sectional view 1000 of a shaft of an RF tissue ablation clamp, such as the distal end of electrosurgical instrument 106 introduced in FIGS. 1-4E, according to one or more aspects of the present disclosure. The cross-sectional view of taken transverse to the longitudinal axis of the elongated longitudinal shaft 110, showing an exemplary internal configuration of the elongated longitudinal shaft 110. As shown, the elongated longitudinal shaft 110 includes lumen 416 (FIG. 4D). Within lumen 416, another shaft 1002 can be included. This can correspond, for example, to the inner shaft 214 (FIG. 2B) and can be open-sided as illustrated in the cross section of FIG. 10A in the shape of a tray with one side absent. The shaft 1002 can include the lumen 414 (FIG. 4D).

FIG. 10B illustrates a shaft seal 1006 (also referred to generally as a plug) that can be used within the lumen 416 as an insufflation seal in a manner that accommodates the shaft 1002. As shown, the shaft seal 1006 includes an opening 1010 to accommodate the general size of the shaft 1002. The shaft seal 1006 further includes cutouts 1008 extending beyond the general opening 1010 to accommodate the tops of the two sides of the shaft 1002 that are left open as shown in FIG. 10A. This shaft seal 1006 can limit gas flow through the lumen 416 (and/or 414). The shaft seal 1006 can be formed from a variety of materials ranging from metals, allows, to various polymeric materials with varying degrees of hardness and flexibility.

The shaft seal 1006 can, in some embodiments, not be flush with the base of the shaft 1002 within the lumen 414 to allow for mechanical and/or electrical connections to extend within the tray formed in the shaft 1002 from the handle 118 (FIG. 1) to the electrodes of jaws 114, 116 (FIGS. 1-4D). In other embodiments, the shaft seal 1006 can include a portion that has a relatively higher degree of hardness and/or inflexibility that can attach for example to an inner surface of the elongated longitudinal shaft 110 away from the base of the tray within shaft 1002. The shaft seal 1006 can include another portion that has a relatively lower degree of hardness and/or greater flexibility (such as a more flexible polymer material) that overlaps any mechanical and/or electrical connections extending along the shaft 1002. The greater flexibility can allow for movement of the connections without disturbing the position or integrity of the shaft seal 1006.

The shaft seal 1006 can be placed at one location along the inner length of the lumen 416 within the elongated longitudinal shaft 110. Alternatively, multiple shaft seals 1006 can be placed along the length within the lumen 416, of one or both alternative configurations of the shaft seal 1006. Other approaches can also or alternatively be used to further increase the level of sealing that occurs within the elongated longitudinal shaft 110. For example, FIG. 10C illustrates an additional member 1010 that can be placed within the general opening 1010 of shaft seal 1006 (e.g., instead of including the flexible portion that might overlap any connections within a tray of shaft 1002). The additional member 1010 can be placed at just the same location as the shaft seal 1006 (e.g., extending for only approximately the overall width of the shaft seal 1006), or can extend along a length within the lumen 414 beyond the shaft seal 1006.

The additional member 1010 can include multiple flexible members 1012 that, together, impede or otherwise prevent insufflation gas from passing therethrough. The flexible members 1012 can extend from a center of the additional member 1010. The additional member 1010 can be, for example, a stiff or moderately stiff shaft with a small diameter (e.g., smaller diameter than the lumen 414) made of metal or plastic (as just a few examples). The flexible members 1012 can be, for example, bristles or other elements that together impede or stop air flow from passing through them.

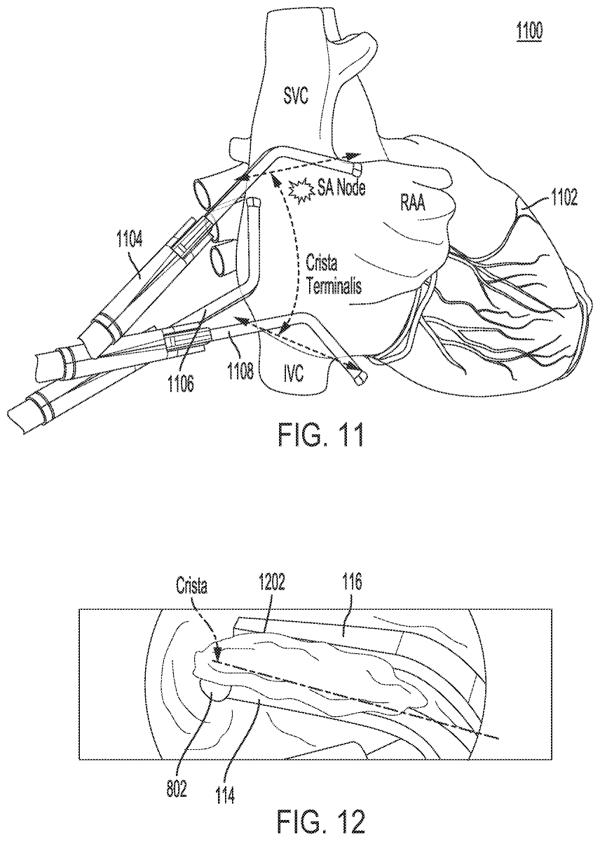

As noted previously, electrosurgical instrument 106 according to the various embodiments of the present disclosure can be used to ablate/treat a variety of tissues, of which cardiac tissue is an example. FIG. 11 illustrates an exemplary side view 1100 of a heart showing candidate ablation locations with an exemplary RF tissue ablation clamp, such as electrosurgical instrument 106, according to one or more aspects of the present disclosure. For example, the candidate ablation locations illustrated in FIG. 11 can correspond to a lesion set useful to treat inappropriate sinus tachycardia, a syndrome in which the sinus heart rate is inexplicably faster than expected and associated symptoms are present. As an example, the heart rate at rest can exceed 100 beats per minute.

To perform the lesion set illustrated in FIG. 11, the electrosurgical instrument 106 can be maneuvered into a first orientation 1106. In orientation 1106, the electrosurgical instrument 106 can be used to create a lesion along the CT line from the inferior vena cava (IVC) to the superior vena cava (SVC), either starting from the IVC and working up to the SVC, or conversely from the SVC to the IVC. The electrosurgical instrument 106, due to the orientation of the jaws 114, 116, the placement of the electrodes 304, 306 on the inner pad surfaces 310, and the gripping surfaces 502, 504, the CT tissue is pinched tangentially along the length of the right atrium between the SVC and IVC. An example of jaws 114, 116 with CT tissue pinched tangentially therebetween is illustrated in FIG. 12. As shown in FIG. 12, epicardial tissue 1202 is pinched between jaws 114, 116 (with atraumatic bumper tip 802) along the CT line.

Next, referring again to FIG. 11, the electrosurgical instrument 106 can be twisted to a second orientation 1104, for example twisted approximately 90 degrees to point the tips of the jaws 114, 116 towards the patient's feet, and go down over the SVC to form an encircling ablation. This is done adjacent the sinoatrial (SA) node, but cannot ablate any tissue of the SA node. The SA node is typically not visually apparent on the epicardial surface of heart 1102, and therefore an electrophysiology (EP mapping system can be used to map the right atrium and can push a catheter to allow a surgeon to see the SA node location. The SA node can further be marked for the duration of the procedure to aid in preventing ablation of the SA node during the procedure. This can be achieved, for example, using a marking tool. The SA node can be marked with a surgical pen, for example, with a patch, and/or with a kittner. Marking can be of the entire area indicated by an activation map (e.g., from the EP mapping system). Examples of ink used can include methylene blue and gentian violet as just some examples.

The first lesion from first orientation 1106 and the second lesion from orientation 1104 can be performed while the electrosurgical instrument 106 is extending into the chest cavity of the patient from a same port. In such examples, a third lesion from a third orientation 1108 can be performed to encircle the IVC to form an encircling ablation via a different port, such as the superior port. Alternatively, all three lesions can be formed via access through the same port. A target result of the ablations from the first orientation 1106, second orientation 1104, and third orientation 1106 can be an ablation line to fully connect the SVC and IVC ablation lines. Should any gaps exist, the surgeon can perform epicardial touchups between lines, and the area can be re-mapped with the EP mapping system to confirm transmurality. Should transmurality not have been established yet, further touch ups can be performed with the electrosurgical instrument 106.

In some embodiments, touch-ups can be performed by the electrodes on the inner surfaces of the jaws 114, 116 of the electrosurgical instrument 106. In other embodiments, the electrosurgical instrument 106 can further include linear electrodes on the outer face of each jaw 114, 116 such that when the jaws 114, 116 are closed, the outer surface (e.g., the base 308 each jaw) can be used like a unidirectional bipolar device. Further, the electrosurgical device 106 can be compatible with the EP mapping system, for example with the EP mapping system software being configured to contain an avatar representation of the electrosurgical device 106's shape and size (e.g., at least the shape and size of the jaws 114, 116) that would show real time position information in relation to the generated map. Further, the electrosurgical device 106 can include pace/sense electrodes in the jaws 114, 116 to allow the surgeon to perform the epicardial mapping before, during, and after creating the lesion set with the pace/sense electrodes.

Beneficially, the lesion set created by the electrosurgical instrument 106 can be performed along intact cardiac atrial tissue during a closed chest, beating heart procedure without use of an accessory such as an endoscopic kittner or dissection of the pulmonary veins. This can be achieved in response to the specific jaw angle of the electrosurgical instrument 106 as discussed above and illustrated in examples in the figures (which can have been optimized, for example, for right atrial VATS access), as well as in response to the textured gripping surfaces of the jaws 114, 116 which facilitate tissue capture and retention in a tangential orientation.

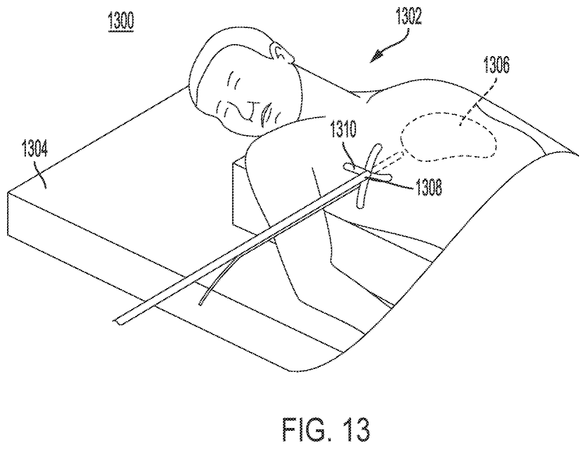

FIG. 13 illustrates an exemplary top perspective view 1300 of a patient 1302 on a surgical surface 1304 showing insertion of the RF tissue ablation clamp into the chest cavity for treating a tissue of a heart 1306 of the patient 1302. For example, this illustrates an example using a minimally invasive procedure using a percutaneous intercostal penetration. One or more percutaneous intercostal penetrations can be made, e.g. Three (one for a port for a camera, one for a port for a working device, such as graspers or kittner(s), and one for the electrosurgical instrument 106), in one or more corresponding access locations 1308. The direction and location of the percutaneous intercostal penetrations will depend on where the cardiac tissue to be treated is located, so that ablation of the cardiac tissue is accessible by the electrosurgical device 106.

For intercostal penetration the lung can be temporarily deflated to allow easier passage of the electrosurgical instrument 106 through the pleural space. One or more suitable access devices can be used to obtain access to the intercostal spaces between the ribs. These devices can be used to protect the incision location and minimize trauma thereto and can include, but are not limited, to trocar sleeves, ports, or other types of percutaneous access cannulae. For example, FIG. 13 illustrates a trocar 1310 (and can also include a tensioning member as well, in some examples).

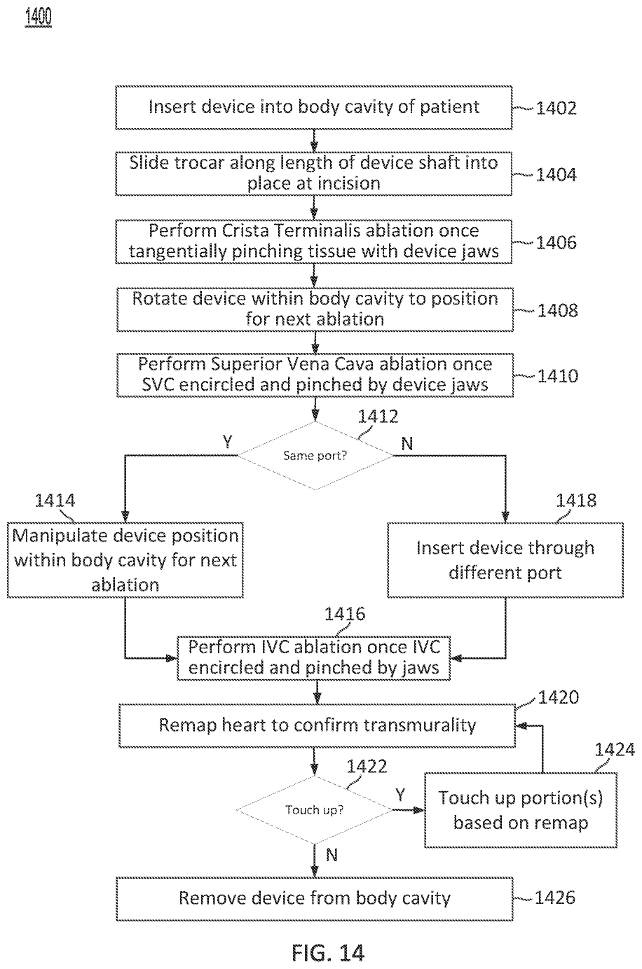

Using the intercostal method, transmural ablations can be performed on other cardiac tissue using RF energy, as previously described herein and below (e.g., FIG. 14). Ablation is repeated until all ablation locations have been treated. An electrosurgical instrument 106 can be inserted into different access locations depending on which directional approach is preferred for the cardiac tissue which requires ablation. Once all areas are treated by the electrosurgical instrument 106, the right lung is re-inflated and the access location can be closed.

FIG. 14 illustrates an exemplary method 1400 of using an exemplary RF tissue ablation clamp in an ablation procedure according to one or more aspects of the present disclosure. Aspects of the method 1400 can be executed by an exemplary RF tissue ablation clamp, such as the electrosurgical instrument 106 utilizing one or more components of the electrosurgical instrument 106 and various combinations thereof. As illustrated, the method 1400 includes a number of enumerated steps, but embodiments of the method 1400 can include additional steps before, during, after, and in between the enumerated steps. Further, in some embodiments, one or more of the enumerated steps can be omitted or performed in a different order.

At block 1402, an electrosurgical instrument 106 is inserted through a small incision into the body cavity of a patient. This can occur, for example, after a trocar incision has been made (e.g., by a thoracic trocar). The incision can be for an intercostal penetration, as discussed above with respect to FIG. 13 as discussed above. The trocar can be removed, e.g. Due to the size of the electrosurgical instrument 106 and specifically the angled geometry of the jaws 114, 116. In some examples, both jaws 114 and 116, hinge mechanism 112, and/or some length of the elongated longitudinal shaft 110 of the electrosurgical instrument 1056 can be inserted at block 1402.

At block 1404, a trocar that had been previously incorporated onto the elongated longitudinal shaft 110 of the electrosurgical instrument 106 can be slid along the length of the elongated longitudinal shaft 110 towards a distal end of the electrosurgical instrument 106 (i.e., towards the incision). This can be done to prevent or impede release of gas from the body cavity of the patient (e.g., where that cavity is insufflated). In other embodiments, block 1404 can alternatively involve a stitch instead of the trocar or in addition thereto.

At block 1406, the electrosurgical instrument 106 can be used to perform ablation of tissue along the Crista Terminalis line. This can be done after first angling the electrosurgical instrument within the cavity so that the distal tips of the jaws 114, 116 are orientated in a generally cranial direction, generally in alignment with the Crista Terminalis of the patient. Once oriented, the user can deploy the trigger of the electrosurgical instrument 106 (e.g., plunger 118) to begin closing the jaws 114, 116.

Because the bases 308 (FIGS. 3A, 3B) are in proximity (e.g., touching) the tissue along the CT line, and in particular because the gripping surfaces 502, 504 (FIGS. 5A-5L) are in contact with the tissue, as the jaw 116 sweeps along the surface of the tissue, the gripping surfaces 502, 504 catch tissue to create a tangential bite of tissue for the ablation between the first and second jaws 114, 116.

At block 1408, the electrosurgical instrument 106 can be rotated within the body cavity of the patient to position the jaws of the electrosurgical instrument 106 for another ablation. For example, after completing the CT line ablation, the electrosurgical instrument can be rotated generally in the transverse plane (e.g., approximately 90 degrees) so that the distal tips of the jaws 114, 116 are now oriented generally in a posterior direction (e.g., with the back of the hinge mechanism 112 in an anterior direction and the distal tips in the posterior direction). In alternative approaches, the electrosurgical instrument 106 can be rotated a different direction such that the back of the hinge mechanism 112 in a lateral direction towards patient's right or left.

At block 1410, the electrosurgical instrument 106 can be used to perform ablation of tissue of the superior vena cava (SVC) once the jaws 114, 116 of the electrosurgical instrument 106 are encircling the SVC. This can include the plunger 118 being depressed again to cause the jaws 114, 116 to close about the SVC for a good grip.

At decision block 1412, if the same port will be used for the third lesion according to embodiments of the present disclosure, then the method 1400 proceeds to block 1414.

At block 1414, the electrosurgical instrument 106's position can be positioned within the body cavity of the patient using the same port through which the first two lesions were performed.

At block 1416, the electrosurgical instrument 106 can be used to perform ablation of tissue of the inferior vena cava (IVC) once the jaws 114, 116 of the electrosurgical instrument 106 are encircling the IVC. This can include the plunger 118 being depressed again to cause the jaws 114, 116 to close about the IVC for a good grip.

Alternatively, returning to decision block 1412, if a different port will be used, then the method 1400 proceeds to block 1418.

At block 1418, the electrosurgical instrument 106 is removed from the current port (e.g., via an inferior port) and inserted into a different port (e.g., a superior port). This can again include, according to embodiments of the present disclosure, using the slidable trocar on the shaft of the electrosurgical instrument. Once inserted and in the vicinity of the IVC, the method 1400 proceeds to block 1416 as discussed above.

At block 1420, the area can be re-mapped, for example with an EP mapping system. As noted previously, a target result of the ablations from blocks 1406, 1410, and 1416 can be an ablation line along the CT to fully connect the SVC and IVC ablation lines. The area can be re-mapped to confirm transmurality. Should any gaps exist, the surgeon can perform epicardial touchups between lines. Should transmurality not have been established yet, further touch ups can be performed with the electrosurgical instrument 106. This is illustrated with blocks 1422-1426 below.

At decision block 1422, if any gaps do exist, or touch-ups are otherwise determined to be necessary, then the method 1400 proceeds to block 1424.

At block 1424, the electrosurgical instrument 106 can be used to perform touch-up (e.g., re-ablation after positioning in accordance with the discussion above) based on the results of remapping. The method 1400 can proceed from block 1424 back to block 1420, where re-mapping can again occur to confirm transmurality. The method 1400 can then proceed as discussed above and further below.

Returning to decision block 1422, if gaps are not identified from the re-mapping, or touch-ups are otherwise not determined to be necessary, then the method 1400 proceeds to block 1426. At block 1426, the procedure ends and the electrosurgical instrument 106 can be removed from the body cavity. In some examples, such as discussed with respect to FIG. 13, once all areas are treated, the right lung can be re-inflated and/or the access location can be closed.

As used herein, including in the claims, “or” as used in a list of items (for example, a list of items prefaced by a phrase such as “at least one of” or “one or more of”) indicates an inclusive list such that, for example, a list of [at least one of A, B, or C] means A or B or C or AB or AC or BC or ABC (i.e., A and B and C).

As those of some skill in this art will by now appreciate and depending on the particular application at hand, many modifications, substitutions and variations can be made in and to the materials, apparatus, configurations and methods of use of the devices of the present disclosure without departing from the spirit and scope thereof. In light of this, the scope of the present disclosure should not be limited to that of the particular embodiments illustrated and described herein, as they are merely by way of some examples thereof, but rather, should be fully commensurate with that of the claims appended hereafter and their functional equivalents.

1. A tissue grabbing apparatus, comprising:
a first jaw comprising a first jaw member approximately parallel to a shaft of the tissue grabbing apparatus and a second jaw member offset from approximately parallel to the shaft, the first jaw further comprising a first surface having a base of the first jaw member and a base of the second jaw member, and a second surface comprising an inner surface of the first jaw member and the second jaw member,
wherein the first jaw further comprises at least one electrode extending along the second surface including at least part of the first jaw member and the second jaw member, and a first grasping surface comprising a first grasping pattern extending along the second surface including at least part of the first jaw member and the second jaw member and along at least part of the first surface;
a second jaw comprising a third jaw member at an angle to the shaft, and a fourth jaw member offset from parallel to the third jaw member while the tissue grabbing apparatus is in an open position, the second jaw further comprising a third surface having a base of the third jaw member and a base of the fourth jaw member, and a fourth surface facing the second surface and comprising an inner surface of the third jaw member and the fourth jaw member,
wherein the second jaw further comprises at least one electrode extending along the fourth surface including at least part of the third jaw member and the fourth jaw member, and a second grasping surface comprising a second grasping pattern extending along the fourth surface including at least part of the third jaw member and the fourth jaw member and along at least part of the third surface; and
a hinge assembly connecting the first and second jaws and configured to move the second jaw relative to the first jaw in a direction transverse to an axis parallel to the shaft between the open position and a closed position where the second jaw is approximately parallel to the first jaw and the shaft.

2. The tissue grabbing apparatus of claim 1, wherein:
the first jaw further comprises a fifth surface interfacing between the first surface and the second surface, the fifth surface also comprising the first grasping pattern, and
the second jaw further comprises a sixth surface interfacing between the third surface and the fourth surface, the sixth surface also comprising the second grasping pattern.

3. The tissue grabbing apparatus of claim 1, wherein the first jaw member and the second jaw member meet at a first apex comprising an angle, and the third jaw member and the fourth jaw member meet at a second apex comprising the angle.

4. The tissue grabbing apparatus of claim 1, wherein the hinge assembly comprises at least one of a bell crank hinge mechanism, a rack-and-pinion hinge mechanism, or a cam hinge mechanism.

5. The tissue grabbing apparatus of claim 1, wherein the first jaw and the second jaw are removeable and replaceable from respective distal ends of the hinge assembly.

6. The tissue grabbing apparatus of claim 1, further comprising:
an atraumatic tip coupled to a distal end of the first jaw.

7. The tissue grabbing apparatus of claim 6, wherein the atraumatic tip comprises a blue color, or a red color, or a white color for increasing visibility of the tip in contrast with surrounding tissues.

8. A surgical clamp system for grabbing and ablating tissue, comprising:
a handle;
an elongated shaft comprising a proximal end coupled to the handle and a distal end;
a jaw assembly comprising:
a first jaw comprising a first surface having a base of the first jaw, a second surface comprising an inner surface of the first jaw, at least one electrode extending along the second surface, and a first grasping surface comprising a first grasping pattern extending along at least part of the first surface and at least part of the second surface; and
a second jaw comprising a third surface having a base of the second jaw, a fourth surface comprising an inner surface of the second jaw, at least one electrode extending along the fourth surface, and a second grasping surface comprising a second grasping pattern extending along at least part of the fourth surface and at least part of the third surface; and
a hinge assembly coupled to the distal end of the shaft, connecting the first and second jaws and configured to move the second jaw relative to the first jaw in a direction transverse to an axis parallel to the shaft.

9. The surgical clamp system of claim 8, further comprising:
a plunger coupled via the handle and shaft to the hinge assembly, such that actuation of the plunger causes the second jaw to move in the direction.

10. The surgical clamp system of claim 8, further comprising:
a trocar slidably coupled around the shaft and configured to provide an insufflation seal to the surgical clamp system after insertion of the jaw assembly into a patient.

11. The surgical clamp system of claim 8, further comprising:
a seal within the shaft configured to provide an internal insufflation seal to the surgical clamp system in use.

12. The surgical clamp system of claim 8, further comprising:
an atraumatic tip coupled to a distal end of the first jaw.

13. The surgical clamp system of claim 8, wherein the hinge assembly comprises at least one of a bell crank hinge mechanism, a rack-and-pinion hinge mechanism, or a cam hinge mechanism.

14. The surgical clamp system of claim 8, wherein the first grasping surface and the second grasping surface each comprises a plurality of pyramid or cuboidially-shaped teeth each culminating in a pointed, flat, chamfered or filleted surface.

15. The surgical clamp system of claim 14, wherein each pyramid-shaped tooth of the first and second grasping surfaces is biased in a direction based on a force profile of the surgical clamp system.

16. A tissue grabbing apparatus comprising:
a first jaw comprising a first surface having a base of the first jaw, a second surface comprising an inner surface of the first jaw, at least one electrode extending along the second surface, and a first grasping surface comprising a first grasping pattern extending along at least part of the first surface and at least part of the second surface; and
a second jaw coupled to the first jaw via a hinge assembly, the second jaw comprising a third surface having a base of the second jaw, a fourth surface comprising an inner surface of the second jaw, at least one electrode extending along the fourth surface, and a second grasping surface comprising a second grasping pattern extending along at least part of the fourth surface and at least part of the third surface,
wherein the first grasping surface and the second grasping surface each comprises a plurality of pyramid-shaped teeth each and comprising a pitch between centerpoints of each pyramid-shaped tooth of between one hundredth of an inch and five hundredths of an inch and a height of each pyramid-shaped tooth of between one hundredth of an inch and two hundredths of an inch.

17. The tissue grabbing apparatus of claim 16, wherein each pyramid-shaped tooth of the first and second grasping surfaces is biased in a direction based on a force profile of the tissue grabbing apparatus.

18. The tissue grabbing apparatus of claim 16, wherein the plurality of teeth are in a cross or diamond grid configuration.

19. The tissue grabbing apparatus of claim 16, wherein the first grasping surface terminates with a first point at an edge of the first grasping surface adjacent to an edge of the second grasping surface, and the second grasping surface terminates with a second point at the edge of the second grasping surface adjacent the edge of the first grasping surface.

20. The tissue grabbing apparatus of claim 16, wherein the hinge assembly configured to move the second jaw relative to the first jaw in a direction transverse to an axis parallel to a shaft of the tissue grabbing apparatus between an open position and a closed position where the second jaw is approximately parallel to the first jaw and the shaft, the first and second grasping surfaces configured to grab tissue as the second jaw moves to the closed position.

21. The tissue grabbing apparatus of claim 1, wherein an angle between an outer surface of the second jaw member and the shaft is about 60 degrees.

22. The tissue grabbing apparatus of claim 1, further comprising a gripping feature on an outer surface of the first jaw and an outer surface of the second jaw.

23. The tissue grabbing apparatus of claim 22, wherein the gripping feature comprises an abrasive coating.

24. The tissue grabbing apparatus of claim 22, wherein the gripping feature comprises a pattern, wherein the pattern is machined, etched, knurled, coined, or stamped into the first jaw and the second jaw.

25. The tissue grabbing apparatus of claim 1, further comprising an insulator component, wherein the insulator component is permanently attached from the first and second jaws via mechanical snap fits.

26. The tissue grabbing apparatus of claim 22, wherein the gripping feature comprises a pattern, wherein the pattern is injection molded into an insulator component coupled to the first and second jaws.

27. The tissue grabbing apparatus of claim 1, further comprising a cam assembly, wherein the cam assembly is configured to slide axially within the jaw assembly to move the second jaw relative to the first jaw.

28. The tissue grabbing apparatus of claim 26, wherein the second jaw comprises at least one bearing, wherein the at least one bearing is configured to slide within a track of the cam assembly.

29. The surgical clamp system of claim 11, wherein the seal comprises a plug component, wherein the plug component reduces a cross-sectional area inside the shaft.

30. The tissue grabbing apparatus of claim 1, wherein the hinge assembly comprises a fixed angle.

31. The tissue grabbing apparatus of claim 30, wherein the fixed angle is about 30 degrees.
